(19) United States
(12) Patent Application Publication
(10) Pub. No.: US 2009/0246145 A1
(43) Pub. Date: Oct. 1, 2009

(54) Imaging Correlates of Neurogenesis With MRI

(76) Inventor: Scott A. Small, New York, NY (US)

Correspondence Address: COOPER & DUNHAM, LLP, 30 Rockefeller Plaza, 20th Floor, NEW YORK, NY, 10112, US

(21) Appl. No.: 12/085,156

(22) PCT Filed: Nov. 14, 2006
(86) PCT No.: PCT/US2006/044392

§ 371 Date: May 14, 2008

Related U.S. Application Data
(60) Provisional application No. 60/736,629, filed on Nov. 14, 2005.

(51) Int. Cl. A61K 49/00; A61K 31/551; A61K 31/519; A61K 31/445; A61K 31/137

(52) U.S. Cl. 424/9.361; 514/220; 514/317; 514/649; 514/259.41

(57) ABSTRACT

This invention provides a method for treating a mammalian subject afflicted with a disorder associated with reduced neurogenesis in the subject's hippocampal dentate gyrus which comprises administering to the subject a therapeutically effective amount of a compound which increases cerebral blood volume in the subject's hippocampal dentate gyrus by a percentage greater than that by which it increases the cerebral blood volume in the subject's hippocampal CA1 region, thereby treating the subject.

This invention was made with support under United States Government Grant No. DAAD19-02-01-0267 from DARPA. Accordingly, the United States Government has certain rights in the subject invention.

Throughout this application, certain publications are referenced. Full citations for Experimental Details I-III, as well as additional related references, may be found immediately following Experimental Details section III. Numerically cited references contained in Experimental Details IV are disclosed at the end of that particular section. The disclosures of these publications are hereby incorporated by reference into this application in order to more fully describe the state of the art as of the date of the invention described and claimed herein.

### BACKGROUND OF THE INVENTION

In the last 6 years, neurogenesis has emerged as a fundamental process underlying CNS physiology and disease. Dr. Gage and co-workers have discovered neurogenesis in the dentate gyrus of human hippocampus, demonstrated that neurogenesis can be regulated, and shown functional neurogenesis in the adult hippocampus (Ray, Peterson et al. 1993; Palmer, Ray et al. 1995; Kempermann, Kuhn et al. 1997; Eriksson, Perfilieva et al. 1998; van Praag, Kempermann et al. 1999; van Praag, Schinder et al. 2002). Contrary to long established dogma, these findings build a compelling case that humans are able to generate new nerve cells throughout their life. This work has opened the door to the possibility of novel therapies for many diseases and disorders of the human CNS and peripheral nervous system.

A number of studies have linked exercise to hippocampal neurogenesis. Studies by Kempermann et al. (1998) have shown that neurogenesis continues to occur in the dentate gyrus of senescent mice and can be stimulated by living in an enriched environment offering social interaction, exploration, and physical activity (Kempermann, Kuhn et al. 1998). Although neurogenesis decreases with increasing age, stimulation through an enriched environment was shown to increase neuronal survival and differentiation. In a subsequent study (van Praag, et al. 1999), running was shown to be more effective than a range of other conditions in increasing neuronal proliferation, survival, and differentiation in adult mice. The other conditions considered were water-maze learning, yoke swimming, and enriched environment, and standard housing.

Activity-dependent regulation of neuronal plasticity and self repair (Kempermann and Gage 2000) is a motivating factor for the use of physical therapies in the treatment of brain injury. In many injuries/diseases, exercise cannot be started early or at all because of the patient's physical condition. The functional outcome of therapeutic intervention is complicated to predict, and depends on a wide range of factors, including the specifics of the disease/injury, family and community resources, and the accuracy of diagnosis. An adjunct to current therapies that induces neurogenesis from early stages of a neurological disease or injury may enhance outcomes to make these patients more functional.

Currently, post-mortem analysis is the only way to determine whether a compound induces neurogenesis. This requirement is obviously prohibitive in determining whether compounds induce neurogenesis in humans. Thus, developing an in vivo indicator of neurogenesis has emerged as an important goal in order to screen, validate, and optimize potential neurogenesis-inducing drugs.

### SUMMARY OF THE INVENTION

This invention provides a method for treating a mammalian subject afflicted with a disorder associated with reduced neurogenesis in the subject's hippocampal dentate gyrus which comprises administering to the subject a therapeutically effective amount of a compound which increases cerebral blood volume in the subject's hippocampal dentate gyrus by a percentage greater than that by which it increases the cerebral blood volume in the subject's hippocampal CA1 region, thereby treating the subject.

This invention also provides a method for inhibiting the onset in a mammalian subject of a disorder associated with reduced neurogenesis in the subject's hippocampal dentate gyrus which comprises administering to the subject a prophylactically effective amount of a compound which increases cerebral blood volume in the subject's hippocampal dentate gyrus by a percentage greater than that by which it increases the cerebral blood volume in the subject's hippocampal CA1 region, thereby inhibiting the onset of the disorder.

This invention further provides a method for treating a mammalian subject afflicted with a disorder associated with increased neurogenesis in the subject's hippocampal dentate gyrus which comprises administering to the subject a therapeutically effective amount of a compound which decreases cerebral blood volume in the subject's hippocampal dentate gyrus by a percentage greater than that by which it decreases the cerebral blood volume in the subject's hippocampal CA1 region, thereby treating the subject.

This invention provides a method for inhibiting the onset in a mammalian subject of a disorder associated with increased neurogenesis in the subject's hippocampal dentate gyrus which comprises administering to the subject a prophylactically effective amount of a compound which decreases cerebral blood volume in the subject's hippocampal dentate gyrus by a percentage greater than that by which it decreases the cerebral blood volume in the subject's hippocampal CA1 region, thereby inhibiting the onset of the disorder.

This invention also provides a method for determining whether an agent increases neurogenesis in a mammalian subject's hippocampal dentate gyrus which comprises (a) determining the cerebral blood volume of a volume of tissue in the subject's hippocampal dentate gyrus and of a volume of tissue in the subject's hippocampal CA1 region; (b) administering the agent to the subject in a manner permitting it to enter the subject's hippocampal dentate gyrus and hippocampal CA1 regions; (c) after a period of time sufficient to permit a detectable increase in neurogenesis in the subject's hippocampal dentate gyrus by an agent known to cause such an increase, determining the cerebral blood volume of the volume of tissue in the subject's hippocampal dentate gyrus and the volume of tissue in the subject's hippocampal CA1 region; and (d) comparing the cerebral blood volumes determined in steps (a) and (c) to determine whether a neurogenesis-specific increase in cerebral blood volume has occurred in the subject's hippocampal dentate gyrus, such increase indicating that the agent increases neurogenesis in the subject's hippocampal dentate gyrus.

This invention further provides a method for determining whether an agent decreases neurogenesis in a mammalian subject's hippocampal dentate gyrus which comprises (a) determining the cerebral blood volume of a volume of tissue in the subject's hippocampal dentate gyrus and a volume of tissue in the subject's hippocampal CA1 region; (b) administering the agent to the subject in a manner permitting it to enter the subject's hippocampal dentate gyrus and hippocampal CA1 regions; (c) after a period of time sufficient to permit a detectable decrease in neurogenesis in the subject's hippocampal dentate gyrus by an agent known to cause such a decrease, determining the cerebral blood volume of the volume of tissue in the subject's hippocampal dentate gyrus and the volume of tissue in the subject's hippocampal CA1 region; and (d) comparing the cerebral blood volumes determined in steps (a) and (c) to determine whether a neurogenesis-specific decrease in cerebral blood volume has occurred in the subject's hippocampal dentate gyrus, such decrease indicating that the agent decreases neurogenesis in the subject's hippocampal dentate gyrus.

### BRIEF DESCRIPTION OF THE FIGURES

FIG. 1: Exercise and CBV in humans. Images: The top image is the pre-contrast MRI from which anatomical landmarks were used to identify ROIs within 4 hippocampal subregions. The middle image shows the ROIs of the 4 hippocampal subregions. Note that the ROIs do not include the borderzones between subregions, which cannot be reliably visualized with MRI. Graphs: Degree of exercise by self report correlated only with CBV from the dentate gyrus as shown in the upper left graph.

FIG. 2: Charts plotting changes in cerebral blood volume (CBV) over time following exercise.

FIG. 3: Design for experiments showing that neurogenesis can be imaged non-invasively with MRI.

FIG. 4: Design for experiments testing series of compounds to determine which compounds induce the most neurogenesis when combined with exercise.

FIG. 5: The correlation between neurogenesis and angiogenesis. Neural precursor cells release a variety of growth factors such as brain derived neurotrophic factor (BDNF), vascular endothelial growth factor (VEGF) and fibroblast growth factor (FGF) that stimulate the vascularization needed to support maturation into neurons. (Reviewed in (Newton and Duman 2004)

FIG. 6: Schematic of the various stages of neural stem cell differentiation and the signaling molecules involved in adult neural stem cell fate decisions.

FIG. 7: Exercise and CBV in humans. Images: The top image is the pre-contrast MRI from which anatomical landmarks were used to identify Regions of Interest (ROI) within 4 hippocampal subregions. The middle image shows the ROIs of the 4 hippocampal subregions. Note that the ROIs do not include the borderzones between subregions which cannot be reliably visualized with MRI. The bottom image is the CBV map, obtained by methods described previously (Small, Chawla et al. 2004). Graphs: Degree of exercise by self-report correlated only with CBV from the dentate gyrus and not with other hippocampal subregions, as shown in the upper left graph.

FIG. 8: Rationale for experimental design to identify changes in dentate gyrus CBV due specifically to neurogenesis.

FIG. 9: Non-invasive high resolution MRI analysis of CBV relies on strict anatomical criteria to identify hippocampal subregions in mice. Top: (left) histochemical identification of hippocampal regions: (right) same as left, with overlay indicating specific regions investigated. Bottom: (left) high resolution MRI of the same area shown in top left; (right) same as bottom left image, with overlay showing specific regions in which CBV was measured.

FIG. 10: A comparison of CBV difference scores in hippocampal subregions between control and exercised groups of mice.

FIG. 11: Correlation between the measured difference in CBV (CBV diff) in the dentate gyrus and the number of newborn neurons (BrdU) without correcting for non-neurogenic effects on CBV (left, correlation coefficient=0.34; p=0.49) and after correcting for non-neurogenic effects on CBV (right, correlation coefficient=0.81, p=0.012). Each point represents the CBV difference score and the total number of BrDU positive cells in the dentate gyrus from a single animal measured after the last scan was performed.

FIG. 12: Selective increases in dentate gyrus CBV observed in exercising mice. (a) The experimental protocol was designed according to the coupling between neurogenesis (blue line) and the delayed formation of new blood vessels (red line). Mice were allowed to exercise for 2 weeks, and BrdU was injected daily during the second week (vertical arrows). Mice were kept alive for 4 more weeks and then processed for post-mortem analyses. MRI was used to generate hippocampal cerebral blood volume (CBV) maps at baseline (week 0) and every 2 weeks thereafter. (b) Exercise had a selective effect on dentate gyrus CBV. Bar graphs show the mean relative cerebral blood volume (rCBV) values for each hippocampal subregion, for the exercise group (black bars) and the non-exercise group (white bars), over the 6-week study. The dentate gyrus was the only hippocampal subregion that showed a significant exercise effect, with CBV peaking at week 4 (left upper graph), while the entorhinal cortex showed a non-significant increase in CBV (c) An individual example, where the left panel shows the high-resolution MRI slice that visualizes the external morphology and internal architecture of the hippocampal formation, the middle panel shows the parcellation of the hippocampal subregions (green=entorhinal cortex, red=dentate gyrus, CA3 subfield dark blue, light blue=CA1 subfield), and the right panel shows the hippocampal CBV map (warmer colors reflect higher CBV).

FIG. 13: Exercise-induced increases in dentate gyrus CBV correlate with neurogenesis. (a) Exercising mice were found to have more BrdU labeling compared to the no-exercise group (left bar graph). As shown by confocal microscopy, the majority of the new cells were NeuN-positive (BrdU labeling=red, NeuN=green, BrdU/NeuN double labeling=yellow). (b) A significant linear relationship was found between changes in dentate gyrus CBV and BrdU labeling (left plot). A quadratic relationship better fits the data (right plot). The vertical stippled line in each plot splits the x-axis into CBV changes that decreased (left of line) versus those that increased (right of line) with exercise.

FIG. 14: Selective increases in dentate gyrus CBV observed in exercising humans. (a) Exercise had a selective effect on dentate gyrus CBV. Bar graph shows the mean relative cerebral blood volume (rCBV) values for each hippocampal subregion, before exercise (white bars) and after exercise (black bars). As in mice, the dentate gyrus was the only hippocampal subregion that showed a significant exercise effect, while the entorhinal cortex showed a non-significant increase in CBV. (b) An individual example, where the left panel shows the high-resolution MRI slice that visualizes the external morphology and internal architecture of the hippocampal formation, the middle panel shows the parcellation of the hippocampal subregions (green=entorhinal cortex, red=dentate gyrus, blue=CA1 subfield, yellow=subiculum), and the right panel shows the hippocampal CBV map (warmer colors reflect higher CBV).

FIG. 15: Exercise-induced increases in dentate gyrus CBV correlate with aerobic fitness and cognition. (a) VO₂max, the gold standard measure of exercise-induced aerobic fitness, increased post-exercise (left bar graph). Cognitively, exercise has its most reliable effect on first-trial learning of new declarative memories (right bar graph). (b) Exercise-induced changes in VO₂max correlated with changes in dentate gyrus (DG) CBV but not with other hippocampal subregions, including the entorhinal cortex (EC) (left scatter plots), confirming the selectivity of the exercise-induced effect. Exercise-induced changes in VO₂max correlated with post-exercise trial 1 learning but not with other cognitive tasks, including delayed recognition (middle scatter plots). Post-exercise trial 1 learning correlated with exercise-induced changes in dentate gyrus CBV (DG CBV), but not with other changes in other hippocampal subregions, including the entorhinal cortex (EC CBV) (right scatter plots).

### DETAILED DESCRIPTION OF THE INVENTION

### Definitions

As used in this application, except as otherwise expressly provided herein, each of the following terms shall have the meaning set forth below.

As used herein, “administering” an agent can be effected or performed using any of the various methods and delivery systems known to those skilled in the art. The administering can be performed, for example, intravenously, intraperitoneally, via cerebrospinal fluid, orally, nasally, via implant, transmucosally, transdermally, intramuscularly, and subcutaneously.

The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA). As used herein, “agent” shall mean any chemical entity, including, without limitation, a protein, an antibody, a nucleic acid, a small molecule, and any combination thereof.

As used herein, “cerebral blood volume” shall mean (i) the volume of blood present in a volume of cerebral tissue, or (ii) a quantitative value (e.g. 1 μm³) correlative either with the volume of blood present in a volume of cerebral tissue and/or with the metabolic activity in that volume of cerebral tissue.

As used herein, “contrast agent” shall mean, where used with respect to brain imaging, any substance administrable to a subject which results in an intravascular enhancement. Examples of contrast agents include paramagnetic substances used in magnetic resonance imaging (such as deoxyhemoglobin or gadolinium).

As used herein, “prophylactically effective amount” means an amount sufficient to inhibit the onset of a disorder associated with a change in neurogenesis in a subject's hippocampal dentate gyrus.

As used herein, “subject” shall mean any animal, such as a human, non-human primate, mouse, rat, guinea pig or rabbit.

As used herein, “therapeutically effective amount” means an amount sufficient to treat a subject afflicted with a disorder associated with a change in neurogenesis in a subject's hippocampal dentate gyrus.

As used herein, “treating” shall mean slowing, stopping or reversing the progression of a disorder.

### Embodiments of the Invention

This invention provides a method for treating a mammalian subject afflicted with a disorder associated with reduced neurogenesis in the subject's hippocampal dentate gyrus which comprises administering to the subject a therapeutically effective amount of a compound which increases cerebral blood volume in the subject's hippocampal dentate gyrus by a percentage greater than that by which it increases the cerebral blood volume in the subject's hippocampal CA1 region, thereby treating the subject.

In one embodiment, the subject is a human. In another embodiment, the disorder is selected from the group consisting of Alzheimer's disease, post-traumatic stress syndrome, age-related memory loss and depression. In one embodiment, the disorder is age-related memory loss, and the subject is older than 65-years old. In another embodiment, the compound is a serotonin-selective uptake inhibitor.

This invention provides a method for inhibiting the onset in a mammalian subject of a disorder associated with reduced neurogenesis in the subject's hippocampal dentate gyrus which comprises administering to the subject a prophylactically effective amount of a compound which increases cerebral blood volume in the subject's hippocampal dentate gyrus by a percentage greater than that by which it increases the cerebral blood volume in the subject's hippocampal CA1 region, thereby inhibiting the onset of the disorder.

In one embodiment, the subject is a human. In another embodiment, the disorder is selected from the group consisting of Alzheimer's disease, post-traumatic stress syndrome, age-related memory loss and depression. In one embodiment, the disorder is age-related memory loss and the subject is older than 65-years old. In another embodiment, the compound is a serotonin-selective uptake inhibitor.

This invention further provides a method for treating a mammalian subject afflicted with a disorder associated with increased neurogenesis in the subject's hippocampal dentate gyrus which comprises administering to the subject a therapeutically effective amount of a compound which decreases cerebral blood volume in the subject's hippocampal dentate gyrus by a percentage greater than that by which it decreases the cerebral blood volume in the subject's hippocampal CA1 region, thereby treating the subject. In one embodiment, the subject is human. In another embodiment, the disorder is epilepsy.

This invention also provides a method for inhibiting the onset in a mammalian subject of a disorder associated with increased neurogenesis in the subject's hippocampal dentate gyrus which comprises administering to the subject a prophylactically effective amount of a compound which decreases cerebral blood volume in the subject's hippocampal dentate gyrus by a percentage greater than that by which it decreases the cerebral blood volume in the subject's hippocampal CA1 region, thereby inhibiting the onset of the disorder. In one embodiment, the subject is human. In another embodiment, the disorder is epilepsy.

This invention provides a method for determining whether an agent increases neurogenesis in a mammalian subject's hippocampal dentate gyrus which comprises (a) determining the cerebral blood volume of a volume of tissue in the subject's hippocampal dentate gyrus and of a volume of tissue in the subject's hippocampal CA1 region; (b) administering the agent to the subject in a manner permitting it to enter the subject's hippocampal dentate gyrus and hippocampal CA1 regions; (c) after a period of time sufficient to permit a detectable increase in neurogenesis in the subject's hippocampal dentate gyrus by an agent known to cause such an increase, determining the cerebral blood volume of the volume of tissue in the subject's hippocampal dentate gyrus and the volume of tissue in the subject's hippocampal CA1 region; and (d) comparing the cerebral blood volumes determined in steps (a) and (c) to determine whether a neurogenesis-specific increase in cerebral blood volume has occurred in the subject's hippocampal dentate gyrus, such increase indicating that the agent increases neurogenesis in the subject's hippocampal dentate gyrus. In one embodiment, determining cerebral blood volume is performed using magnetic resonance imaging. In another embodiment, the cerebral blood volume is determined with respect to a volume of tissue which is 1 mm³ or less, and determining the cerebral blood volume comprises the steps of (a) acquiring a first image of the volume of tissue in vivo; (b) administering a contrast agent to the volume of tissue; (c) acquiring a second image of the volume of tissue in vivo, wherein the second image is acquired at least four minutes after the administration of the contrast agent; and (d) determining the cerebral blood volume of the volume of tissue based on the first and second images. In one embodiment, the contrast agent comprises gadolinium.

In another embodiment, determining the cerebral blood volume with respect to a volume of tissue is performed by a method comprising the steps of (a) acquiring a first magnetic resonance image of the volume of tissue in vivo; (b) administering intraperitoneally to the subject a gadolinium-containing contrast agent in an amount greater than about 1 mg per kg body weight and less than about 20 mg per kg body weight; (c) acquiring a second magnetic resonance image of the volume of tissue in vivo, which second image is acquired at least about 15 minutes after, but not more than about 2 hours after, administering the contrast agent; and (d) determining the amount of cerebral blood volume based on the first and second images. In one embodiment, the contrast agent is gadolinium pentate. In another embodiment, the subject is a mouse or a rat. In yet another embodiment, the agent is a serotonin selective uptake inhibitor.

This invention further provides a method for determining whether an agent decreases neurogenesis in a mammalian subject's hippocampal dentate gyrus which comprises (a) determining the cerebral blood volume of a volume of tissue in the subject's hippocampal dentate gyrus and a volume of tissue in the subject's hippocampal CA1 region; (b) administering the agent to the subject in a manner permitting it to enter the subject's hippocampal dentate gyrus and hippocampal CA1 regions; (c) after a period of time sufficient to permit a detectable decrease in neurogenesis in the subject's hippocampal dentate gyrus by an agent known to cause such a decrease, determining the cerebral blood volume of the volume of tissue in the subject's hippocampal dentate gyrus and the volume of tissue in the subject's hippocampal CA1 region; and (d) comparing the cerebral blood volumes determined in steps (a) and (c) to determine whether a neurogenesis-specific decrease in cerebral blood volume has occurred in the subject's hippocampal dentate gyrus, such decrease indicating that the agent decreases neurogenesis in the subject's hippocampal dentate gyrus. In one embodiment, determining cerebral blood volume is performed using magnetic resonance imaging.

In another embodiment, the cerebral blood volume is determined with respect to a volume of tissue which is 1 mm³ or less, and determining the cerebral blood volume comprises the steps of (a) acquiring a first image of the volume of tissue in vivo; (b) administering a contrast agent to the volume of tissue; (c) acquiring a second image of the volume of tissue in vivo, wherein the second image is acquired at least four minutes after the administration of the contrast agent; and (d) determining the cerebral blood volume of the volume of tissue based on the first and second images. In one embodiment, the contrast agent comprises gadolinium.

In another embodiment, determining the cerebral blood volume with respect to a volume of tissue is performed by a method comprising the steps of (a) acquiring a first magnetic resonance image of the volume of tissue in vivo; (b) administering intraperitoneally to the subject a gadolinium-containing contrast agent in an amount greater than about 1 mg per kg body weight and less than about 20 mg per kg body weight; (c) acquiring a second magnetic resonance image of the volume of tissue in vivo, which second image is acquired at least about 15 minutes after, but not more than about 2 hours after, administering the contrast agent; and (d) determining the amount of cerebral blood volume based on the first and second images. In one embodiment, the contrast agent is gadolinium pentate. In another embodiment, the subject is a mouse or a rat.

### Supplemental Embodiments

The following embodiments relate to the gadolinium-based MRI methods discussed above.

In a further embodiment, the amount of the gadolinium-containing contrast agent is administered in an amount of about 10 mg per kg body weight. In another embodiment, the second magnetic resonance image is acquired about 45 minutes after administering the gadolinium-containing contrast agent.

This invention also provides the above-described method further comprising the step of intraperitoneally administering a saline solution to the subject, which administering follows either step (c) or step (d).

In one embodiment, the subject is a mouse and at least about 4 ml of saline solution is administered. In another embodiment, the subject is a mouse and about 5 ml of saline solution is administered. In yet another embodiment, the subject is an animal model for a human neurological disease.

This invention provides a method for determining the change in the amount of blood in a volume of cerebral tissue (cerebral blood volume) in a mammalian subject over a predefined period of time, comprising determining the cerebral blood volume at a plurality of time points during the predefined period of time and comparing the cerebral blood volumes so determined, so as to determine the change in the cerebral blood volume over the predefined period of time, wherein at each time point, determining the cerebral blood volume is performed according to the above-described method, with the proviso that at each time point other than the final time point in the predefined period of time, a saline solution is intraperitoneally administered to the subject following either step (c) or step (d).

In one embodiment, the predefined period of time is one month or longer. In another embodiment, the predefined period of time is six month or longer. In yet another embodiment, the predefined period of time is one year or longer. In a further embodiment, the predefined period of time is two years or longer.

In one embodiment, the plurality of time points during the predefined period of time number 3 or more. In another embodiment, the plurality of time points during the predefined period of time number 5 or more.

In yet another embodiment, the plurality of time points during the predefined period of time number 10 or more. In a further embodiment, the plurality of time points during the predefined period of time number 20 or more.

This invention is illustrated in the Experimental Details section which follows. This section is set forth to aid in an understanding of the invention but is not intended to, and should not be construed to limit in any way the invention as set forth in the claims which follow thereafter.

### EXPERIMENTAL DETAILS I

### Background and Significance

The dentate gyrus is a rare and privileged brain region in that it maintains the capacity for neurogenesis throughout the life span. Since the dentate gyrus is involved in cognitive function the ability to stimulate neurogenesis may be harnessed as a way to prevent cognitive deficits caused by sleep deprivation. Work in rodents suggests that physical exercise is a potent stimulant of dentate gyrus neurogenesis. Currently, documenting neurogenesis requires sacrificing animals and performing post-mortem analysis on brain slices. This requirement is obviously prohibitive in humans, and accounts for why it still remains unknown whether exercise stimulates neurogenesis in the human dentate gyrus. With this limitation in mind, an MRI approach was recently developed that relies on the tight spatial and temporal coupling between neurogenesis and angiogenesis. Angiogenesis results in an increase in cerebral blood volume (CBV), a parameter which can be measured with MRI, even within the small dimensions of the dentate gyrus.

### Preliminary Studies

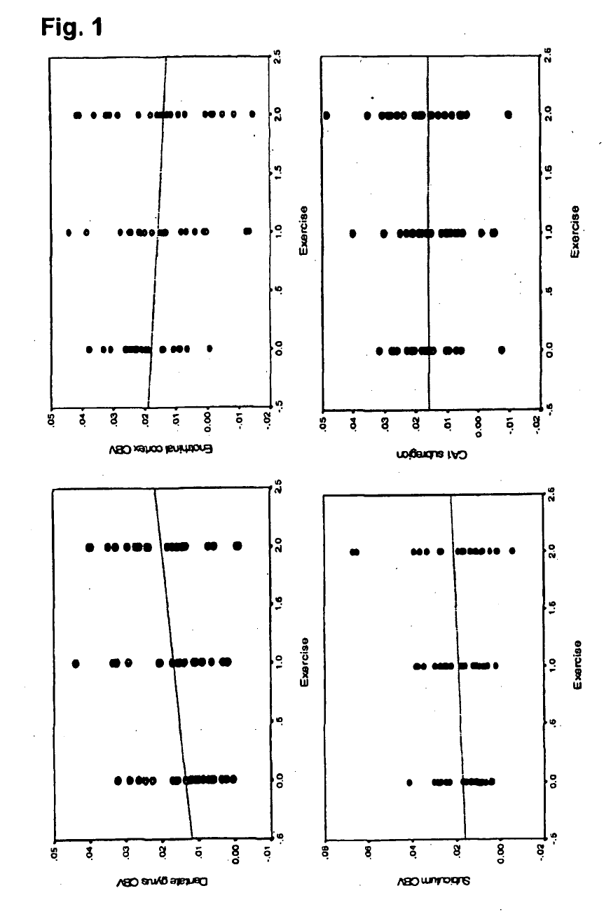

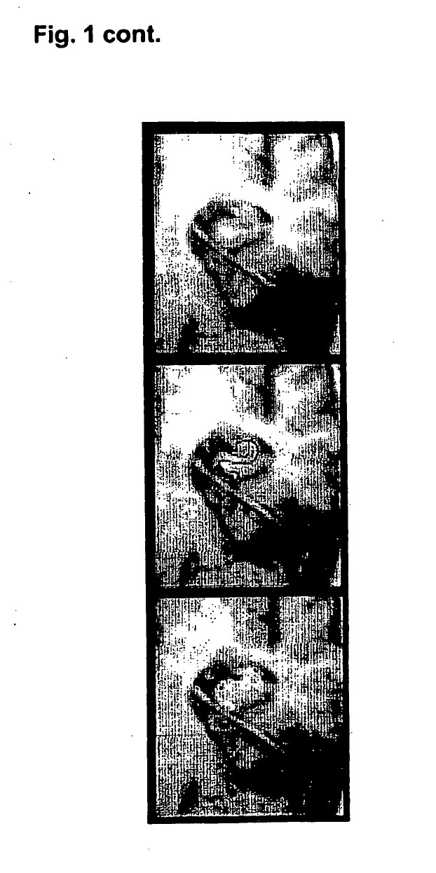

As part of a large scale epidemiological study, 66 subjects were administered an exercise questionnaire in which they answered yes/no to the following questions: “Have you gone out for a walk in the last month?”, and, “Have your performed physical exercise for physical conditioning in the last month?”. Each positive answer was assigned a +1 and so subjects could have a total score ranging from 0-2. All subjects were imaged with an MRI protocol used to estimate CBV from the four hippocampal subregions—the entorhinal cortex, the dentate gyrus, CA1 and the subiculum (as shown in FIG. 1). A correlational analysis revealed that only the CBV measured from the dentate gyrus correlated with self report of exercise, as shown in FIG. 1.

Although the results were supportive of a relationship between exercise and dentate gyrus CBV, this study has a number of significant limitations. First, the questions were limited in their scope. Second, questionnaires in general are fraught with many of the subjective inaccuracies that come with self-reporting. Third, CBV was measured at a single time point, and there are many other factors which may covary with self-reporting of exercise, and thus it cannot be concluded that exercise per se accounts for dentate gyrus CBV. These concerns are best addressed by actually quantifying the amount of exercise during a month, and by looking for a change of CBV before and after exercise.

### Research Methods and Design

Subjects

Twenty subjects, 20-45 years of age, are recruited from the Columbia University/New York Presbyterian Hospital community. Subjects are sedentary, habitual non-exercisers, who qualify as below average fitness by American Heart Association (AHA) standards (VO₂max<43 for men, <37 for women). All subjects are nonsmokers. Subjects are recruited by flyers posted throughout the Columbia-Presbyterian Medical Center. After phone screening to determine eligibility, subjects perform an incremental exercise test on a cycle ergometer.

Experimental Groups

Group I: Moderate intensity exercise: Subjects are permitted to select from a series of aerobic activities, e.g., cycling on a stationary ergometer, running on a treadmill, climbing on a Stairmaster, or using an elliptical trainer.

The exercise program is based on the subject's fitness assessment. Specifically, subjects start their initial exercise at a heart rate equivalent to 55-65% of their maximum heart rate obtained during the VO₂max test. Subjects exercise at this intensity for two weeks, after which the intensity was maintained at 65% of maximum HR for the remainder of the 12-week training program. This moderate intensity training elicited increases in VO₂max of approximately 8-10%.

Group 2: High intensity exercise: Again, subjects are permitted to select from a series of aerobic activities and for weeks 1 and 2 of the 12 week program, they train at 55-65% of maximum HR. In weeks 3 and 4, the intensity is increased to 65-75% of maximum HR and in weeks 5-12, the intensity is increased to 75% of maximum HR. This high intensity training program elicits increases in VO₂max of approximately 15%.

Both training programs are 12 weeks in length. A trainer is available for each subject to ensure that exercise is conducted at the proper intensity level. Adherence to the training program is documented by weekly logs and by computerized attendance records at the facilities and by data from HR monitors used during each training session. Subjects are contacted on a weekly basis by research staff to monitor their progress.

After completion of the exercise programs, subjects return for follow-up VO₂max and RRV testing. Data collection staff are blind to group assignment. All training sessions in both conditions consist of 10-15 minutes of warm-up and cool down and 30-40 minutes of intense workout. These sessions are carried out 4 days/week. Training programs are conducted in the PlusOne Fitness Center on the Columbia medical school campus. Superb cooperation is attained with PlusOne staff in a previous study.

In order to assure quality control and adherence, subjects complete detailed logs of their activity during each training session. These logs contain information on the date and duration of exercise training and the activities of each training session. Throughout all training sessions, subjects wear Polar heart rate monitors that record HR throughout the session. These data are downloaded after each session and evaluated on a weekly basis. This assists in subject adherence and provided rigorous documentation of training intensity levels.

Cardiovascular Indices

Aerobic Capacity: Maximum aerobic fitness (VO₂max) is measured by a graded exercise test on an Ergoline 800S electronic-braked cycle ergometer (SensorMedics Corp., Anaheim, Calif.). Each subject begins exercising at 30 watts (W) for two minutes, and the work rate is increased continually by 30 W each two minutes until VO₂max criteria (RQ of 1.1 or >, increases in ventilation without concomitant increases in VO2, maximum age-predicted heart rate is reached and or volitional fatigue) is reached. Minute ventilation is measured by a pneumotachometer connected to a FLO-1 volume transducer module (PHYSIO-DYNE Instrument Corp., Quogue, N.Y.). Percentage of expired oxygen (O²) and carbon dioxide (CO²) is measured using a paramagnetic O² and infrared CO² analyzers connected to a computerized system (MAX-1, PHYSIO-DYNE Instrument Corp., Quogue, N.Y.). These analyzers are calibrated against known medical grade gases. The highest VO₂ value attained during the graded exercise test is considered VO₂max. Identical test procedures are carried out at the end of the training program to determine changes in VO₂max.

Cardiac Autonomic Modulation: Continuous measures of ECG, blood pressure, and respiration are recorded during 10-min resting periods in both the seated and supine positions. ECG electrodes are placed on the right shoulder, on the left anterior axillary line at the 10th intercostal space and in the right lower quadrant. Analog ECG signals are digitized at 500 Hz by a National Instruments 16 bit A/D conversion board and passed to a microcomputer. The ECG waveform is submitted to an R-wave detection routine implemented by custom-written event detection software, resulting in an RR interval series. Errors in marking of R-waves were corrected interactively.

For both the supine and seated 10-min resting periods, mean RRI, and the following indices of RRV are computed: the standard deviation of the RR interval (SDRR), the root mean squared successive difference (rMSSD), and spectral power in the low (0.04-0.15 Hz (LF)) and high (0.15-0.50 Hz (HF)) frequency bands. The spectra of these series are calculated on 300 second epochs using an interval method for computing Fourier transforms similar to that described by DeBoer, Karamaker, and Strackee (deBoer, 1984). Prior to computing Fourier transforms, the mean of the RR interval series is subtracted from each value in the series and the series then was filtered using a Hanning window and the power, i.e., variance (in msec²), over the LF and HF bands was summed. Estimates of spectral power are adjusted to account for attenuation produced by this filter.

Respiration

Chest and abdominal respiration signals are collected by a Respitrace monitor. These signals are submitted to a specially written respiration scoring program which produces minutes by minute means of respiratory rate.

MRI

All subjects receive two MRI's, once at baseline and a second MRI at the end of the exercise period.

Generating CBV Maps with MRI

Two sets of oblique coronal 3D T1-weighted images (TR=20 ms; TE=6 ms; flip angle=25 degrees; in plane resolution=0.86 mm×0.86 mm; slice thickness=4 mm) are acquired—the first acquired before and the second acquired 4 minutes after IV administration of a standard dose of Omniscan (0.1 mmol/kg). Slices are oriented perpendicular to the hippocampal long axis, identified on a scout T1-weighted sagital series. The subject is requested to be careful so as not to move between the two images acquisitions.

Acquired images are transferred to Dr. Small's laboratory, and processing is performed on a dual-processor (2.4 GHz Xeon) linux (RedHat7.3) workstation, using image display and analysis software packages (MEDx Sensor Systems). An investigator blinded to subject grouping performs all imaging processing. The AIR program is used to co-register the images. The short acquisition time of the runs enhances the goodness-of-fit of the algorithm. Two methods are used to assess the goodness-of-fit of the motion correction, and are used as criteria for accepting or rejecting a particular study: First a Gnu plot is employed post-correction. If there is a shift of greater than 1 pixel dimension over the scanning time period in any direction in space the study is rejected. Second, two motion-corrected images are subtracted from each other. If there is large signal detected in the residual image, the study is rejected. Only one of the preliminary studies performed is rejected for failing these goodness-of-fit criteria.

The pre-contrast image is subtracted from the post-contrast image, and the difference in the sagittal sinus is recorded. The subtracted image is then divided by the difference in the sagittal sinus and multiplied by 100 yielding absolute CBV maps.

Identifying the Dentate Gyrus and Other Hippocampal Subregions

Among the series of oblique coronal images, it is consistently found that a slice anterior to the lateral geniculate nucleus and posterior to the uncus provides optimal visualization of hippocampal morphology and internal architecture. This slice is the standard slice used for all studies. As shown in FIG. 1, the external morphology of the hippocampus is traced, and a single tracing of the internal morphology follows the hippocampal sulcus and the internal white matter tracts. ROIs of the four subregions of the hippocampal formation are then identified relying on the following anatomical criteria: a) Entorhinal cortex—the lateral and inferior boundary follows the collateral sulcus; the medial boundary is the medial aspect of the temporal lobe; the superior boundary is the hippocampal sulcus and gray/white distinction between subiculum and entorhinal cortex, b) Subiculum—the medial boundary is the medial extent of the hippocampal sulcus and/or the horizontal inflection of the hippocampus; the inferior boundary is the white matter of the underlying parahippocampal gyrus; the superior boundary is the hippocampal sulcus: the lateral boundary is the a few pixels medial to the vertical inflection of the hippocampus, c) CA1 subregion—the medial boundary is 2-3 pixels lateral to the end of the subiculum ROI, approximately at the beginning of the vertical inflection of the hippocampus, and the extension of the hippocampal sulcus/white matter tracts; the inferior boundary is the white matter of the underlying parahippocampal gyrus; the superior boundary is the top of the hippocampal formation, d) Dentate gyrus—the medial boundary is the medial extent of the temporal lobe; the inferior/lateral boundary is the hippocampal sulcus/white matter tracts; the superior boundary is the top of the hippocampal formation, where the alveus is typically identified. Standard atlases are used to identify these anatomical landmarks;

Data Analysis

A range of parameters are recorded, many of which can be used as indicators of individual variance in exercise. For statistical parsimony, VO₂max is used first since it is considered one of the ‘gold-standards’ in the field. ‘CBV difference scores’ are derived by subtracting the last CBV measured from each hippocampal subregion from the first CBV. Because all hippocampal subregions are interconnected as part of a unified physiologic circuit, a multivariate step-wise linear regression analysis is performed, where VO₂max was included as the dependent variable and the four CBV differences scores (from each hippocampal subregion) are included as the independent variables. Demographic variables are included in the model as needed. Although a number of subregions may have demonstrated an exercise-related increase in CBV, an increase in dentate gyrus CBV may have best correlated with an index of exercise. A range of other parameters are explored for use as indices of individual variance in exercise.

### EXPERIMENTAL DETAILS II

### Imaging Neurogenesis in the Dentate Gyrus of Living Humans

### Background and Significance

Against scientific dogma, there is now clear evidence that neurogenesis continues throughout the life-span in select brain region—most notably the dentate gyrus, a primary subregion of the hippocampal circuit. Moreover, manipulations that reliably induce neurogenesis have been identified, such as exercise or serotonin-reuptake inhibitors. The next important step is to determine whether and how neurogenesis influences cognition. Currently, neurogenesis can only be detected in post-mortem tissue, and thus the correlation between neurogenesis and cognition can only be accomplished in non-human animals. The goal of the current project is to develop an imaging technique that can detect, and even quantify, neurogenesis in the dentate gyrus of living humans.

Among all imaging modalities—CT, PET, SPECT, MRI—only MRI (magnetic resonance imaging) has sufficient spatial resolution to visualize the dentate gyrus. MRI-based techniques that rely on intracellular contrast agents to label new-born neurons are under exploration. Although appropriate for animal models, relying on intracellular contrast agent—which requires invasive administration and may interrupt neuronal function—is not on option for detecting neurogenesis in living humans.

Neurogenesis is tightly coupled to angiogenesis, and therefore inducing neurogenesis in the dentate gyrus increases regional cerebral blood volume (CBV). It has been shown that MRI can be used to detect and quantify CBV changes in the dentate gyrus in humans, monkeys, and mice, and that this can be accomplished with complete safety. The primary goal of this project, therefore, is to determine whether CBV changes measured with MRI can detect neurogenesis.

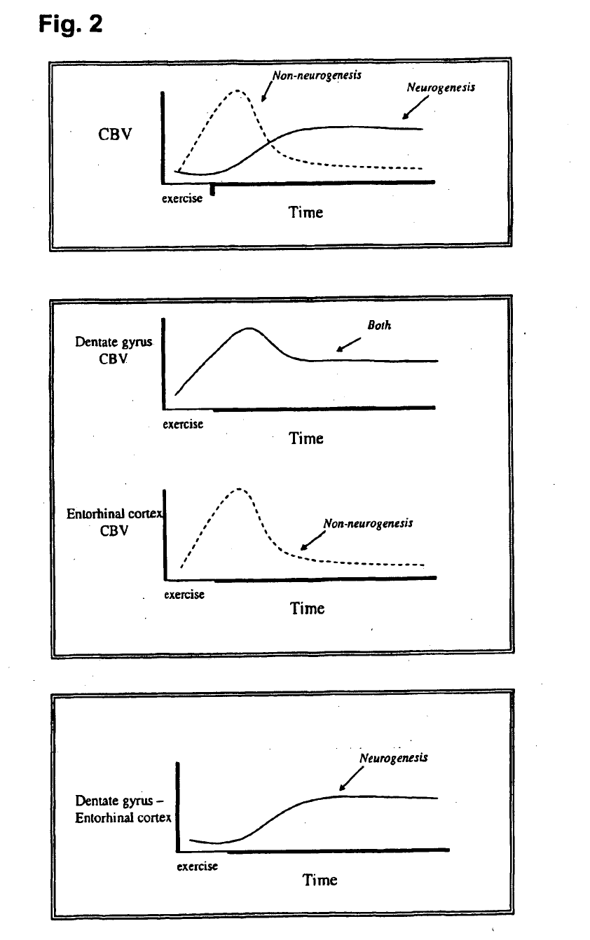

CBV is not selectively coupled to neurogenesis, and other factors such as cardiac output and synaptic activity will influence regional CBV, independent of neurogenesis. Since exercise is expected to modulate these other factors, the question remains on how one can we be sure that a detected change in CBV reflects neurogenesis. The answer lies in spatiotemporal profiles of CBV changes: As shown in the upper panel of FIG. 2, non-neurogenesis factors that influence CBV are expected to peak early and dissipate quickly at the end of an exercise period, in contrast to neurogenesis whose effect on CBV is expected to peak later and remain elevated longer.

Furthermore, the non-neurogenesis factors are expected to occur in the dentate gyrus as well as in other subregions of the hippocampal formation—the entorhinal cortex, the CA3 and CA1 subfields, and the subiculum. Thus, as shown in the middle panel of FIG. 2, it is expected that the CBV curve in the dentate gyrus reflects both non-neurogenesis and neurogenesis factors, while the CBV curve in the other hippocampal subregions reflects only neurogenesis factors. By subtracting the latter CBV curve from the former, it is expected that a CBV curve will be generated that reflects only neurogenesis, as shown in the lower panel of FIG. 2.

Neurogenesis can be Imaged Non-Invasively with MRI

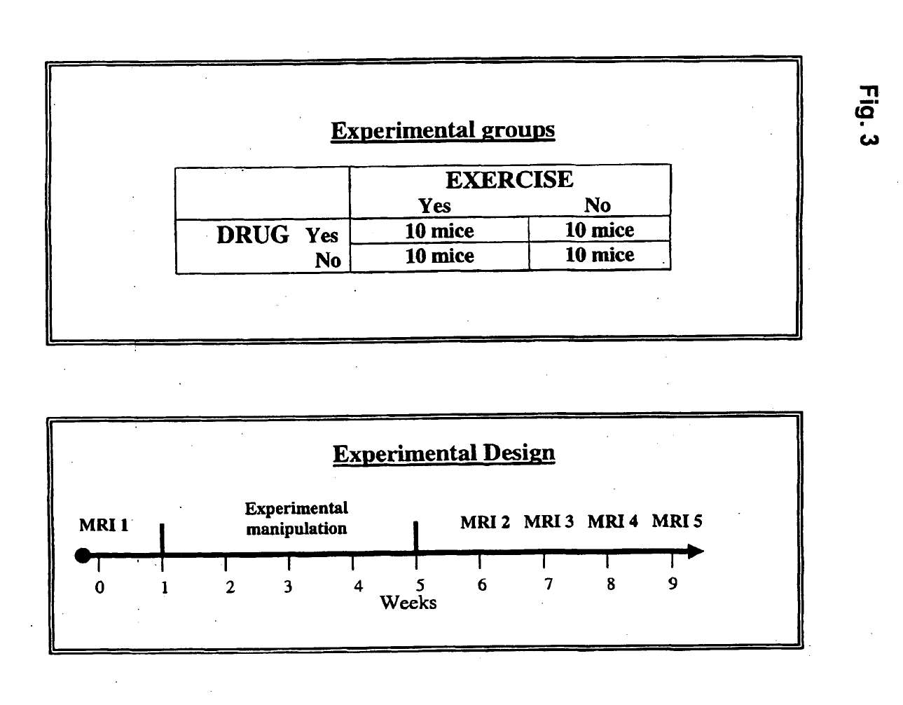

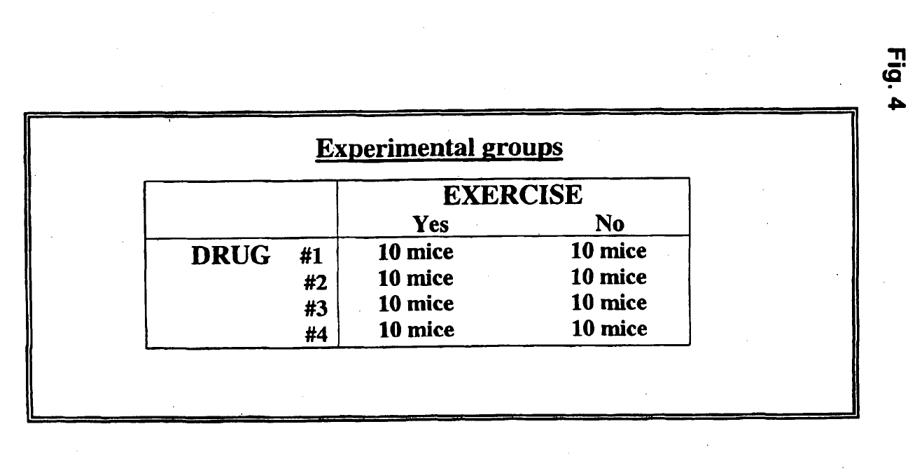

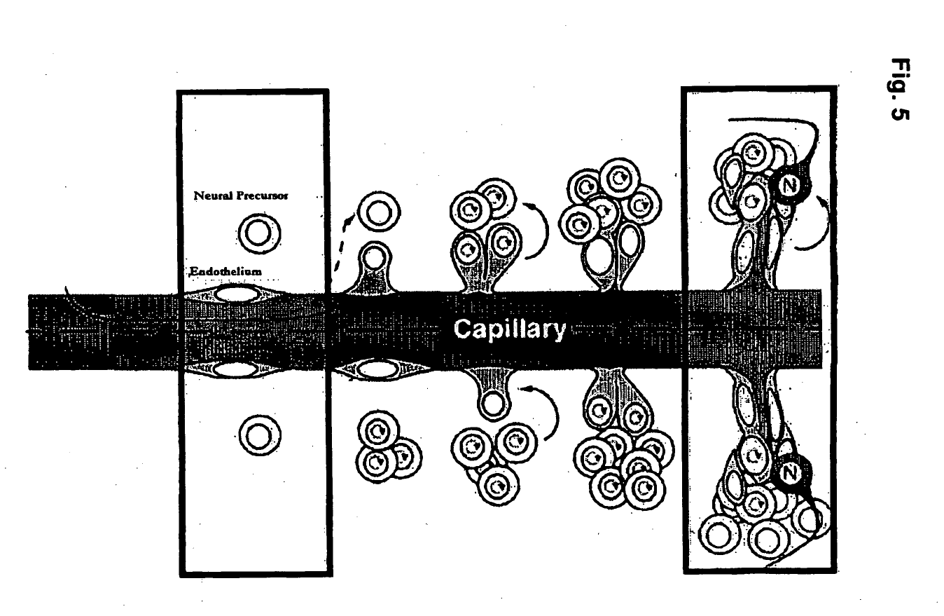

As shown in FIG. 3, four groups of mice are imaged. All mice receive BRDU injections at time zero, and receive their baseline MRI. Each group receive one of four experimental manipulations—exercise with drug, sham-exercise with drug, exercise with placebo, and sham-exercise with placebo. CBV curves are established in the dentate gyrus as well as in other hippocampal subregions—the CA3 and CA subfields, the subiculum, and the entorhinal cortex. The average CBV curve from the other hippocampal subregions are subtracted from the CBV curve generated from the dentate gyrus.

Testing a Series of Different Compounds to Determine which Induces the Most Neurogenesis when Combined with Exercise

Four groups of mice are imaged, following the identical experimental design as discussed above. Results from the four groups are compared using a MANOVA model to determine which drug results in the most neurogenesis.

Testing the Most Neurogenic Drug in Healthy Humans

The experimental groups and experimental design outlined above is replicated with 40 healthy humans as subjects (10 subjects per experimental group).

### EXPERIMENTAL DETAILS III

### Summary

The dentate gyrus is a privileged brain region that maintains the capacity for neurogenesis throughout life. Drugs that accelerate neurogenesis hold great promise as therapeutic agents against many diseases—including Alzheimer's disease, traumatic brain injury, developmental disorders, and stroke. The ability to safely visualize correlates of neurogenesis with imaging techniques is required to screen and validate potential neurogenesis-inducing drugs. Toward this goal, an MRI approach to visualize correlates of neurogenesis in the dentate gyrus will be investigated. The approach is based on the tight spatial and temporal coupling between neurogenesis and angiogenesis. Angiogenesis results in an increased cerebral blood volume (CBV), and CBV is a parameter that has been successfully imaged with MRI from the dentate gyrus of humans, monkeys, and rodents. Preliminary data suggests that CBV in the dentate gyrus of humans and mice is selectively correlated with exercise, a known behavioral modifier of neurogenesis. The goal of this project is to validate this MRI approach by administering neurogenesis-inducing drugs in rats. By systematically mapping the effect of drug in the dentate gyrus as well as in neighboring hippocampal subregions which do not undergo neurogenesis, a pattern of MRI changes that is both sensitive and specific to neurogenesis will be extracted. Once validated in rats, this MRI approach can then be translated to humans for the screening and validation of neurogenesis-inducing drugs.

Recent scientific discoveries indicate that the process of birth, proliferation, and development of new brain neurons can continue at all stages of human life. This study aims to develop an assay for discovering new drugs to stimulate this process. These drugs will provide a new therapeutic strategy for patients suffering from neurological disorders and diseases, including stroke, traumatic brain injury, brain tumors, developmental disorders, and Alzheimer's disease.

Until recently, brain disease and injury were considered to result in permanent loss of neurons with no possibility of cellular regeneration. Extensive evidence now suggests that certain brain areas retain the capability to generate new neurons into adulthood in rodents, nonhuman primates, and humans. These findings point to new approaches for therapy, namely, the pharmacological induction of endogenous neurogenesis. The therapy would have relevance for neurological diseases and injuries, including stroke/ischemia, traumatic brain injury, brain tumors, developmental disorders, and Alzheimer's disease.

### Background and Significance

In the last 6 years, neurogenesis has emerged as a fundamental process underlying CNS physiology and disease. Dr. Fred Gage and co-workers have discovered neurogenesis in the dentate gyrus of human hippocampus, demonstrated that neurogenesis can be regulated, and shown functional neurogenesis in the adult hippocampus (Ray, Peterson et al. 1993; Palmer, Ray et al. 1995; Kempermann, Kuhn et al. 1997; Eriksson, Perfilieva et al. 1998; van Praag, Kempermann et al. 1999; van Praag, Schinder et al. 2002). Contrary to long established dogma, these findings build a compelling case that humans are able to generate new nerve cells throughout their life. This work has opened the door to the possibility of novel therapies for many diseases and disorders of the human CNS and peripheral nervous system.

A number of studies have linked exercise to hippocampal neurogenesis. Studies by Kempermann et al. (1-998) have shown that neurogenesis continues to occur in the dentate gyrus of senescent mice and can be stimulated by living in an enriched environment offering social interaction, exploration, and physical activity (Kempermann, Kuhn et al. 1998). Although neurogenesis decreases with increasing age, stimulation through an enriched environment was shown to increase neuronal survival and differentiation. In a subsequent study (van Praag, et al. 1999), running was shown to be more effective than a range of other conditions in increasing neuronal proliferation, survival, and differentiation in adult mice. The other conditions considered were water-maze learning, yoke swimming, an enriched environment, and standard housing.

Activity-dependent regulation of neuronal plasticity and self repair (Kempermann and Gage 2000) is a motivating factor for the use of physical therapies in the treatment of brain injury. In many injuries/diseases, exercise cannot be started early or at all because of the patient's physical condition. The functional outcome of therapeutic intervention is complicated to predict, and depends on a wide range of factors, including the specifics of the disease/injury, family and community resources, and the accuracy of diagnosis. An adjunct to current therapies that induces neurogenesis from early stages of a neurological disease or injury may enhance outcomes to make these patients more functional.

Currently, post-mortem analysis is the only way to determine whether a compound induces neurogenesis. This requirement is obviously prohibitive in determining whether compounds induce neurogenesis in humans. Thus, developing an in vivo indicator of neurogenesis has emerged as an important goal in order to screen, validate, and optimize potential neurogenesis-inducing drugs. With this goal in mind, during the last few years Dr. Scott Small's laboratory has explored different imaging approaches for visualizing neurogenesis in living subjects.

One approach is the use of MRI-sensitive reporter molecules—analogous to BrdU—that upon injection are incorporated into newly dividing cells. Although in principle these reporter molecules can be developed, a preliminary analysis raised a number of safety concerns regarding this approach. First, the reporter molecule needs to penetrate two natural barriers, the blood-brain barrier and the cell membrane. Even if this first concern can be addressed, the second concern is that the reporter molecule will in all likelihood need to accumulate in high concentrations to achieve favorable signal-to-noise, which might have a deleterious effect on neuronal function. Thus, although an MRI-sensitive neurogenesis reporter molecule may succeed in animal models, it has been concluded that this approach will be problematic when translated to humans because of safety concerns. Despite these concerns MRI-sensitive reporter molecule for mapping neurogenesis are continuing to be explored.

At the same, however, a second approach for visualizing neurogenesis is being explored, which if validated will readily translate to human investigation. This approach is based on the tight spatial and temporal coupling between neurogenesis and angiogenesis summarized in FIG. 5 (Palmer, Willhoite et al. 2000; Louissaint, Rao et al. 2002). Angiogenesis results in a relative increase in regional cerebral blood volume (CBV), and CBV is a parameter that can be measured with MRI (Gonzalez, Fischman et al. 1995). A number of studies have demonstrated that MRI estimations of CBV can detect angiogenesis in living rodents (Lin, Sun et al. 2002; Dunn, Roche et al. 2003; Dunn, Roche et al. 2004; Jiang, Zhang et al. 2005) and indeed a number of studies have shown that MRI measures of CBV can capture changes associated with hippocampal dysfunction and with global measures of brain injury. Over the last few years, MRI-based protocols were developed that can safely measure CBV in hippocampal subregions—including the dentate gyrus—in humans, monkeys, and mice (Small, Wu et al. 2000; Small, Tsai et al. 2002).

Altering the concentration of intravascular contrast agents is the typical approach taken to estimate regional cerebral blood volume (CBV) with MRI (as formally discussed in (Belliveau, Rosen et al. 1990; Kuppusamy, Lin et al. 1996; van Zijl, Eleff et al. 1998; Wu, Wong et al. 2003). Depending on their properties, contrast agents will either affect T1-weighted or T2-weighted signal intensity. By injecting a bolus of gadolinium and tracking the dynamic change in T2*-weighted signal over time Belliveau and colleagues introduced the first MRI approach to measure CBV (Belliveau, Rosen et al. 1990). By plotting signal amplitude against time, the “area under the curve” of the first pass of contrast—the first and heaviest flow of contrast through a specific brain region—can be used to calculate a region's CBV. Dynamic susceptibility contrast (DSC) MRI is typically performed with echo-planar imaging since high temporal resolution is required to capture the transient first pass. This temporal requirement compromises spatial resolution, and DSC cannot, for now, visualize individual hippocampal subregions.

Haake, Lin and colleagues have introduced an alternative gadolinium-based approach that can map CBV with high-spatial resolution (Kuppusamy, Lin et al. 1996; Lin, Paczynski et al. 1997; Lin, Celik et al. 1999). Instead of racing after the first pass of contrast with rapid imaging, CBV measurements are generated from the steady-state T1-weighted changes induced by the contrast agent. Compared to dynamic measurements, steady-state measurements can generate CBV maps with much higher spatial resolution. Indeed, the steady-state CBV approach can achieve the required submillimeter resolution, and can therefore visualize individual hippocampal subregions in humans and monkeys (Small, Chawla et al. 2004).

Both gadolinium and iron oxide particles have been used to map CBV in rodents, typically relying on T2-weighted changes in signal intensity (van Bruggen, Busch et al. 1998; Mandeville, Jenkins et al. 2001; Dunn, Roche et al. 2003; Dunn, Roche et al. 2004; Jiang, Zhang et al. 2005). A variant of the gadolinium based approach has recently been introduced. The main novelty is that gadolinium is introduced via IP (intraperitoneal) rather than IV injections, which is much less traumatic and increases the odds that CBV changes can be mapped repeatedly and safely in the same animal. Aside from this practical difference, conceptually this approach is nearly identical to previous approaches. It was found that this IP approach generates estimates of CBV that are quantitatively similar to IV injections of either gadolinium or iron oxide particles. In related work, Jiang et al (Jiang, Zhang et al., 2005) map CBV relying on T2-weighted signal changes in response to gadolinium (thus very similar to the above-cited approach). They show that this CBV map can indeed detect the emergence of angiogenesis coupled to neurogenesis induced by injecting neuronal progenitor cells.

The next section will review preliminary data suggesting that exercise—an established inducer of neurogenesis—accounts for the variance of CBV measured selectively from the dentate gyrus, and showing that neurogenic compounds using in vitro histological assays can be identified. Lacking, however, is a systematic analysis showing that CBV determined by MRI directly correlates with neurogenesis measured in vitro, using exercise or pharmacological agents as neurogenesis stimulators.

The overall aim of this proposal is to provide further evidence that CBV measured by MRI is a sensitive correlate of neurogenesis. Although other approaches are under development as in vivo indicators of neurogenesis, the significant advantage of the CBV approach is that it is readily translatable to humans. The approach that has been developed for CBV mapping in rodents is nearly identical to the approach currently used in humans. It has been shown that this approach can map CBV in individual hippocampal subregions of the human hippocampus, including the dentate gyrus. Using this approach, CBV mapping is safe, not only for a single time-point measurements but also when used repeatedly over time. Thus, longitudinal experiments can be performed, with imaging before and after drug delivery—where each individual acts as their own control—which is potentially a powerful approach for evaluating drug efficacy.

### Preliminary Studies

Identification of Neurogenic Compounds

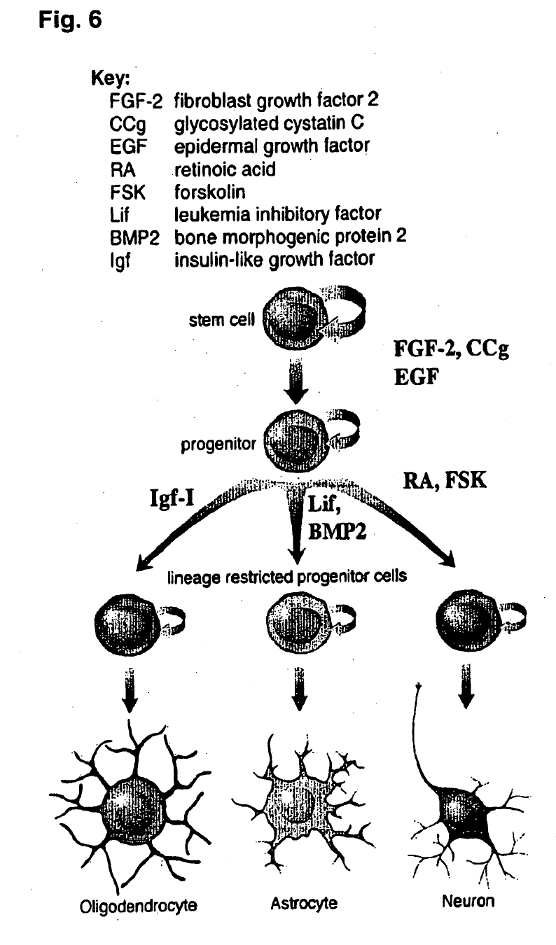

Pioneering studies by a number of laboratories have identified the adult hippocampal neural stem cell (NSC) and the factors that regulate its survival and fate choice determination. These studies have shown that exogenous factors can regulate the process of neurogenesis in vitro. The stages of NSC differentiation and the factors that govern each stage are summarized in FIG. 6.

Cultured rNSCs have been established by Gage, et al., as an in vitro model of neurogenesis in the brain based on their ability to propagate while maintaining stem cell properties (Palmer, Ray et al. 1995). These properties include the ability to self-renew and differentiate into all neural lineages: neurons, oligodendrocytes, and astrocytes. The in vitro results have been corroborated via in vivo transplantation of cultured rNSCs and demonstration that they retain the full range of neurogenic properties (Ray, Peterson et al. 1993; Song, Stevens et al. 2002; van Praag, Schinder et al. 2002; Hsieh, Aimone et al. 2004).

BrainCells' focus is the development of new neurogenesis-based therapeutics, based on enabling technologies developed by Dr. Gage, a co-founder of the company. These technologies and tools form the bases for a neurogenesis platform that enables profiling and selection of drug candidates to promote endogenous neurogenesis for the treatment of CNS disorders.

CBV and Neurogenesis

### CBV and Exercise in Humans

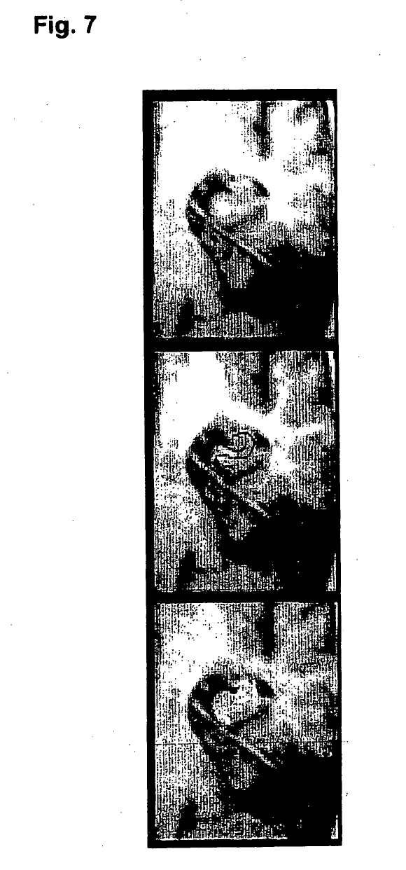

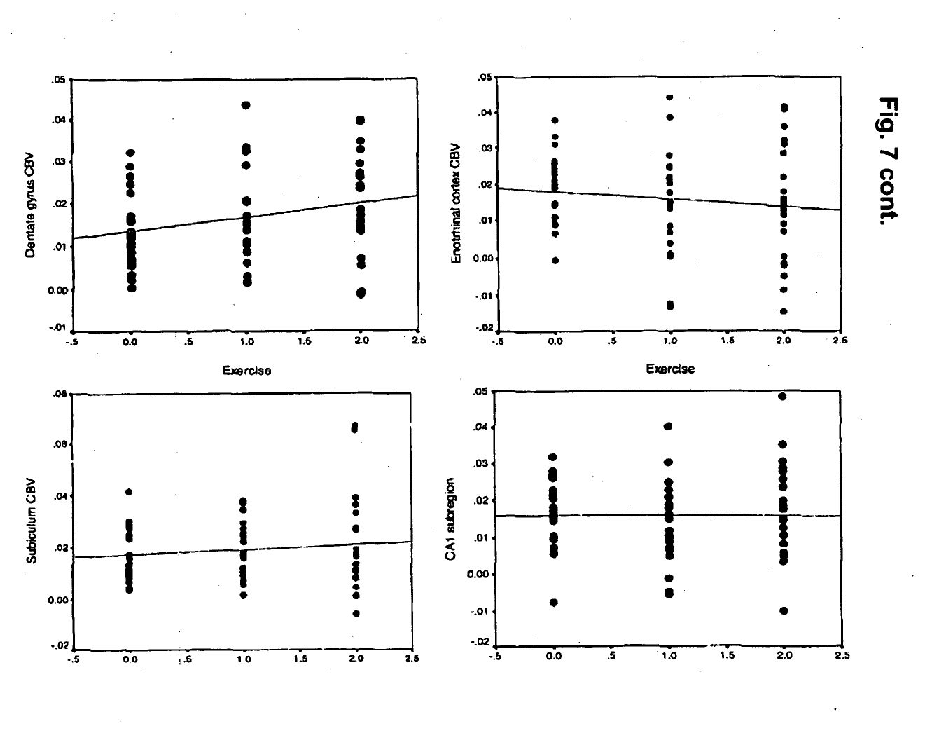

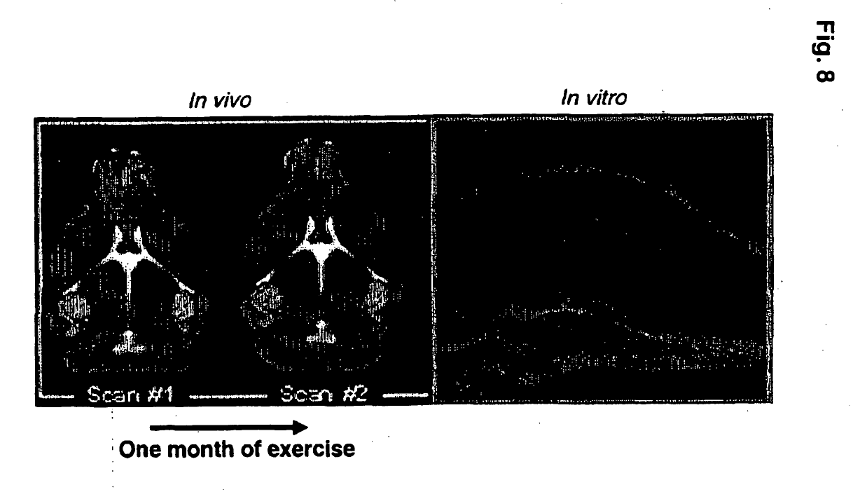

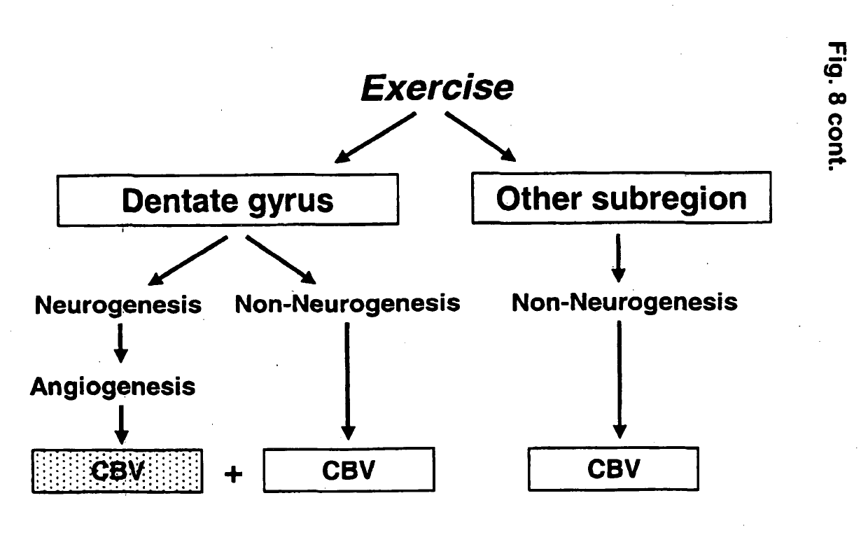

As part of a large scale epidemiological study, 66 subjects were administered an exercise questionnaire in which they answered yes/no to the following questions: “Have you gone out for a walk in the last month?”; and, “Have your performed physical exercise for physical conditioning in the last month?”. Each positive answer was assigned a +1; subjects could have a total score ranging from 0-2. All subjects were imaged with an MRI protocol used to estimate CBV from the four hippocampal subregions—the entorhinal cortex, the dentate gyrus, CA1 and the subiculum (FIG. 7). This protocol was a modification of T1-weighted technique first developed by Lin and Haacke (Lin, Paczynski et al. 1997; Lin, Celik et al. 1999). Gadolinium was administered by IV injection and CBV estimates were derived based on steady-state changes in T1-weighted signal. The modification to the technique was to optimize for visualization of hippocampal subregions. This method has been used to image non-human primates (Small, Chawla et al. 2004).

A correlational analysis revealed that of the hippocampal subregions measured, only the CBV measured from the dentate gyrus correlated with self report of exercise, as shown in FIG. 7. Although the results were supportive of a relationship between exercise and dentate gyrus CBV, this study has a number of significant limitations. First, the questions were limited in their scope and imprecise. Second, questionnaires in general are fraught with many of the inaccuracies that come with self-reporting. Third, CBV was measured at a single time point, and there are many other factors which may covary with self-reporting of exercise, and thus it cannot be concluded that exercise per se accounts for dentate gyrus CBV. These concerns are best addressed by actually quantifying the amount of exercise during a month, and by looking for a change of CBV before and after exercise. This goal motivated the mouse experiments described in the next section.

Correlating Regional CBV and Neurogenesis in Mice

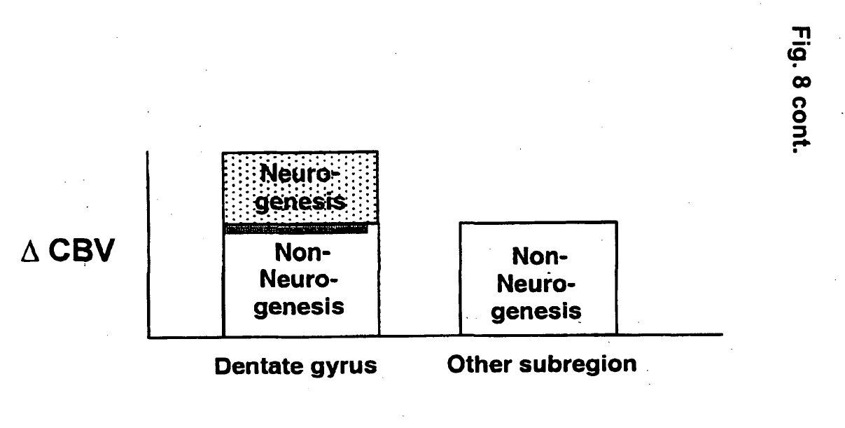

In preliminary studies, the correlation between estimates of CBV changes in individual hippocampal subregions (measured in vivo by MRI) and neurogenesis in mice (measured in vitro histologically) has been evaluated. The rational for the experimental design is represented schematically in FIG. 8.

Because, as noted previously, neurogenesis is coupled with angiogenesis, and angiogenesis is coupled with CBV, the assumption can be made that CBV will be a sensitive marker of neurogenesis. However, because CBV is affected by non-neurogenesis factors, it cannot be assumed that directly measured CBV changes in the dentate gyrus would be specific to neurogenesis.

Ways to impose specificity on CBV measurements were explored in a preliminary set of experiments. An inducer of neurogenesis such as exercise will affect CBV in the dentate gyrus through both a neurogenesis and a non-neurogenesis mechanism. Consequently, if one were to measure a change in CBV before and after exercise, the observed change would be a composite of neurogenesis and non-neurogenesis factors. Thus, the question is how to extract only the neurogenesis contribution from the observed CBV.

Preliminary studies have tested the following assumption: that the non-neurogenic effect of exercise on CBV will be manifest in neighboring hippocampal subregions that do not have neurogenesis capabilities. If this assumption is correct, i.e., that non neurogenesis effects in other regions are equivalent to those in the dentate gyrus, they can be subtracted from the observed CBV to estimate neurogenesis-only CBV effects in the dentate gyrus.

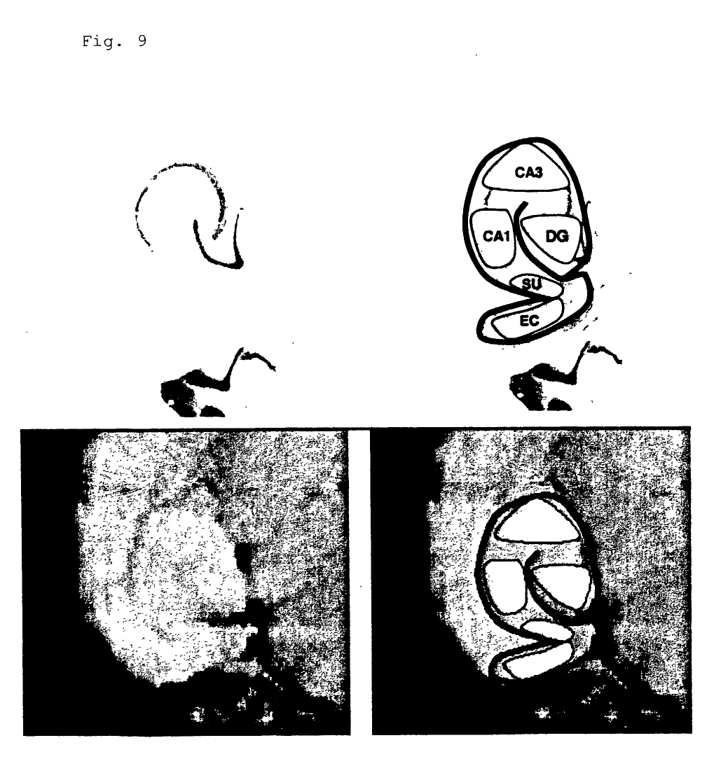

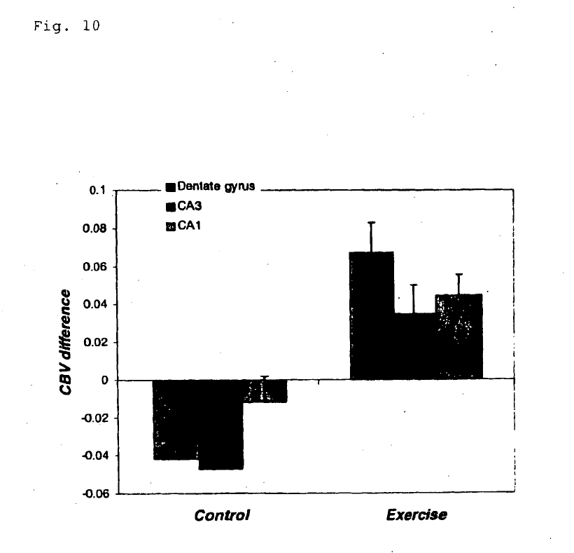

Clearly, this assumption might not be true, and furthermore, it cannot be predicted a priori which of the multiple hippocampal subregions would be most effective in this approach. Therefore, to test this assumption, an experiment was designed in which CBV was estimated in a variety of hippocampal subregions (FIG. 9) using the T2-weighted approach (Moreno, Hua et al. 2005) (attached as an appendix); and multiple linear regression analysis (MLRA) was used to determine which region yielded the best results.

Initial CBV estimates were done in both test and control groups. After a month of exercise for the test group, CBV estimates were repeated for both groups. At this point, all mice were sacrificed, and BrdU labeling was used to quantify hippocampal neurogenesis.

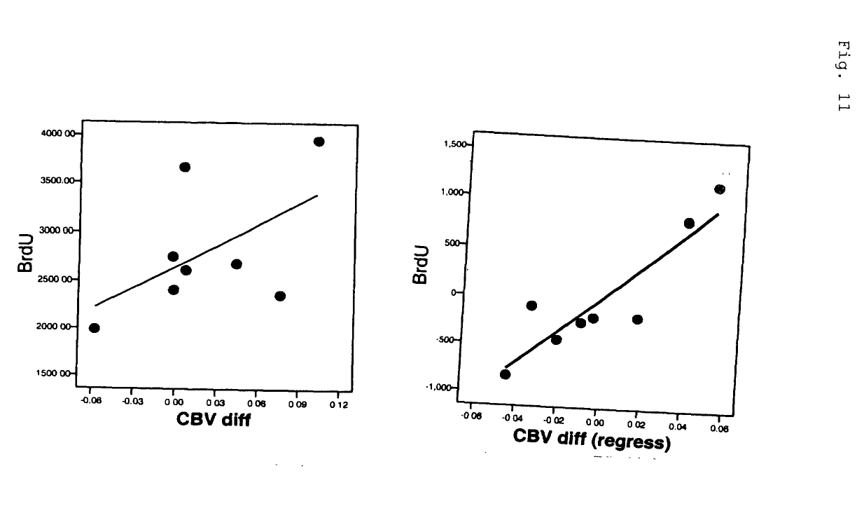

CBV difference scores were derived by subtracting the initial regional CBV estimate from that found after a month with or without exercise. Some of the results are shown in FIG. 10. In the three hippocampal subregions shown, a numerical CBV score increase in the exercising mice versus those that did not exercise was noticed. Although the control group had a decline in CBV score, this decrease was not statistically different from zero. Using a multivariate ANOVA, a between-group difference was only found in the dentate gyrus.

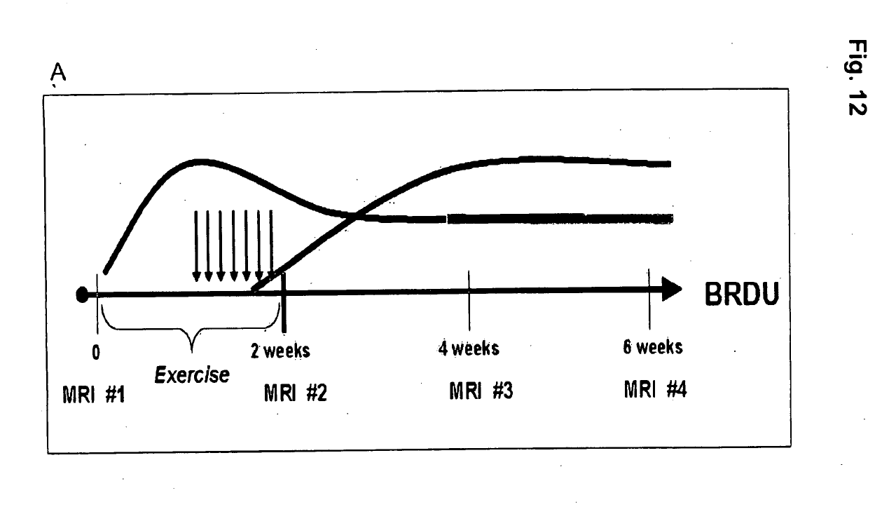

In order to test the starting assumption that a hippocampal subregion outside the dentate gyrus might be used to extract the neurogenesis-specific component of change in CBV, multiple linear regression analyses (MLRA) of the raw data was performed. However, it was not known, a priori, which hippocampal subregion would be most useful (if any), and MLRA allowed for the exploration of options. The result showed that including the CBV difference score for CA1 as a covariant in the analysis resulted in a significant correlation between dentate gyrus CBV and BrDU labeling (shown in right hand plot of FIG. 11).

The left graph of FIG. 11 shows CBV difference (CBV_{exercise} minus CBV_{control}) in the dentate gyrus cross correlated with BrdU neurogenesis measurements. This does not take into account the change in CBV that is due to exercise but not arising from neurogenesis. Note that a positive trend is observed but it is not statistically significant. The graph on the right shows the same correlation, but the dentate gyrus CBV difference has been corrected by subtracting the CBV difference found for the CA1 subregion. This correction yields a statistically significant correlation between changes in dentate gyrus CBV and neurogenesis.

These preliminary results 1) confirm the assumption that it is possible to impose specificity on CBV as a correlate of neurogenesis, and 2) identify which of the hippocampal subregions provides the best estimate of non-neurogenesis exercise induced changes in CBV.

### Research Design and Methods

The specific aims are:
1. Determine the correlation between exercise-induced neurogenesis in rat dentate gyrus and changes in CBV measured by MRI.
2. Determine if compounds with known in vivo neurogenic activity (valproic acid and fluoxetine) can enhance CBV.

For both aims, the approach was similar to that presented in the preliminary results. CBV in hippocampal subregions was measured by MRI in both control and test groups of rats. Neurogenesis in the test groups were stimulated by exercise or by treatment with valproic acid and fluoxetine. Multivariate linear regression analyses was performed to determine the best method for correlating neurogenesis-induced changes in dentate gyrus CBV with histologically measured neurogenesis. The technical details of the experimental methods are provided in the sections below.

CBV Derivations with MRI

### Rodent MRI Lab

The laboratory contains a Bruker AVANCE 400WB spectrometer (Bruker NMR, Inc., Bilerica, Mass.) with an 89 mm-bore 9.4 tesla vertical Bruker magnet (Oxford Instruments Ltd., UK) using a birdcage RF probe and a shielded gradient system up to 100 G/cm. The diameter of the bore and the tesla strength provide stable, very high-resolution images with favorable signal-to-noise. The center also houses a surgery room that contains a dissecting microscope, surgical tools, and anesthetic agents and equipment.

Physiologic Monitoring

Many physiological processes can influence MRI signal in the brain, particularly when measuring resting signal. For this reason the laboratory has a series of physiologic monitoring devices that tightly monitor a range of physiologic measures while the mouse is being imaged. O₂ and CO₂ are continuously monitored with a micro-capnometer; heart rate and pulse rate are continuously monitored using pulse oximetry. Temperature is continuously monitored with a thermistor. An EKG and respiratory rate are recorded if needed through devices built into the magnet.

Anesthesia

Although the heads of the rats are mechanically held in place, head motion has to be minimized with anesthesia; Furthermore, anesthesia reduces the fear and anxiety induced by the scanner. In principle any anesthetic is capable of influencing brain physiology and therefore all anesthetic agents will influence MRI signal; choosing the correct agent, therefore, needs to be done with care. Isoflurane gas (induction phase 3 vol % and maintenance 1.5 vol % at 1 L/min air flow, via a nose cone) was used. The most important advantage of isoflurane over other anesthetic agents is that isoflurane produces none or minimal cerebral hemodynamic changes. CBV relies on hemodynamic coupling—the biophysical relation between oxygen metabolism and cerebral blood flow. It turns out that several anesthetics produce uncoupling, which would be a devastating effect for the experiments. Given this critical consideration, the effects of a variety of anesthetics on T2 signal have been explored. Finally, isoflurane was decided on, although other anesthetic combinations such as ketamaine/xylazine have a similar profile to isoflurane.

Data Acquisition

Three scout scans are first acquired to position the subsequent T2 weighted images along the standard anatomical orientations in a reproducible manner. T2 weighted axial images are acquired with multislice fast spin echo (FSE) sequence using TR/TE_{eff}=200 ms/80 ms, rapid acquisition with relaxation enhancement (RARE) factor=16, FOV=26 mm, acquisition matrix=256×256, slice thickness 0.6 mm, slice gap=0.1 mm and NEX 28. The in-plane resolution is 100 μm. This sequence is repeated 4 times, for a total imaging time of 60 minutes. The first 15 minutes correspond to pre-gadolinium image, after this time period a delay of 1-2 minutes precede the ip gadolinium injection while the mouse is being imaged. The injection lasts 30 seconds. All images are acquired utilizing the same dynamic range, so there is no risk of rescale.

Contrast Delivery

An intravascular contrast agent is required to generate a CBV map of the brain. Different contrast agents have been used for CBV mapping in rodents. Most studies to date have relied on intravenous injections for contrast delivery. Because IV delivery is often problematic in rodents, associated with frequent morbidity and even occasional mortality, it is not ideally suited for longitudinal studies imaging rodents repeatedly over time. Motivated by this concern, an IP protocol using gadolinium was optimized as the contrast agent This protocol has recently been submitted for publication and is supplied with this proposal as an appendix (Moreno, Hua et al. 2005).

Gadolinium (gadodiamide) sterile aqueous solution at a concentration of 287-mg/ml pH between 5.5-7.0 is injected undiluted via a catheter with an OD of 0.6 mm, which is placed intraperitonealy before imaging. The catheter is secure with 6.0 silk suture materials. Once initial images are acquired (pre-contrast), gadolinium is injected IP with a dose of 10 mMol/Kg. After the imaging session is completed, rodents still under anesthesia are injected slowly IP with 2 ml of normal saline solution. As noted in the appendix, it was found that this is required in order to wash out the remaining gadolinium; this was realized empirically since re-imaged animals without this procedure had low contrast to noise ratio (CNR).

Several doses of IP gadolinium were tested. Above 10 mMol it has toxic effects (mainly transient unsteady gait, possibly vertigo) and below 5 mMol Delta R2 values are low. Time course curves allowed us to identify the appropriate interval between gadolinium injection and post contrast imaged (45 minutes).

Imaging Processing

After data reconstruction the raw images were sent to a Linux-based workstation loaded with the MEDx image analysis software package (Sensor System). An investigator blinded to subject grouping did all imaging processing.

CBV maps were generated in accordance with an approach first developed by Li, et al. First, pre- and post-gadolinium images were coregistered. Second, post-gadolinium images were subtracted from pre-gadolinium images. Third, a ‘signal change score’ was determined in a region that contains 100% blood. Although in humans the sagital sinus is used for this determination, in rats the jugular vein is more easily visualized and was for this determination. Fourth, the subtracted images were divided by the change score in the jugular vein yielding CBV maps (Lin, Paczynski et al. 1997).

Regional of interests (ROI) were identified from the anatomical maps of the 5 hippocampal subregions—the entorhinal cortex, the dentate gyrus, the CA1 and CA3 subfields, and the subiculum. Note that identifying the precise border zones between the subregions requires special histological staining, which of course were not available during in vivo imaging. The absence of anatomical landmarks defining the precise boundaries among subregions prevents a volumetric analysis of the subregions; however, as in slice electrophysiology, it is possible to rely on visualized anatomical landmarks to identify the general locale of each subregion. Two landmarks are required to segment the hippocampal formation—its external morphology and identification of the hippocampal fissure. The external morphology of the hippocampal formation can be easily visualized in both T2 and T2*-weighted images. The hippocampal fissure is typically closed in mature living animals; fortunately, the intrahippocampal long vein follows the course of the hippocampal fissure, and veins are readily visualized in T2 and T2*-weighted images. These images were used to identify the hippocampal fissure. Among the series of acquired axial slices, it is possible to successfully identify a ‘single best slice’ in which these anatomical landmarks are most readily visualized. This slice is typically acquired through the middle body of the hippocampal formation (as shown in FIG. 9). Once the anatomical landmarks were identified, a standard mouse brain atlas was used to draw ROIs in each of the hippocampal subregions. The ROI was drawn within the centroid of each subregion, purposefully staying away from borderzones. ROIs were drawn from both the left and the right hippocampal subregions. Previous studies have found that the ROIs across groups were approximately the same size. However, ROI size was monitored and corrected if a systematic difference was observed.

The average CBV from each hippocampal ROI was determined. Finally, “CBV difference-scores” were calculated by subtracting CBV measures from the pre-neurogenic stimulation scan from the CBV measures of the post-exercise scan. These CBV difference-scores were used as the primary variables for the correlational analysis as described below in the “Data Analysis” section.

Exposure to Neurogenic Stimulation Protocol

Male F344 rats age 6-8 weeks (150-250 grams) were housed individually. Animals were divided into control and test groups. The control group was housed in standard cages. There were a total of three test groups over 2 years. There were a minimum of 12 animals per group and the aim for group size was generally 14 per group. All rats received one daily IP injection of 100 m/kg BrdU for 7 consecutive days beginning the first day of treatment (day 1). All animals were analyzed by MRI for determination of CBV at day 1 and day 28. After the completion of the MRI imaging on day 28, the anesthetized animals were sacrificed by transcardial perfusion with 4% paraformaldehyde. The animal brains were removed for post mortem analysis of neuronal proliferation, survival, and differentiation as described in ‘Postmorem analysis’.

### Exercise Test Group (Test Group 1)

The first test group was housed in an activity cage with an activity wheel, with computer monitoring of the wheel's use.

### Drug Treated Test Groups 2 and 3

The second and third test groups were housed similarly to control animals but were treated with known neurogenic compounds for 28 days during the MRI analysis. After the completion of the MRI study, animals were euthanized and perfusion fixed brains were removed and sent to BrainCells Inc. for analysis as described in ‘Postmorem analysis’. Two compounds were proposed for this purpose: valproic acid and fluoxetine.

Valproic acid (VPA; 2-propylpentanoic acid) is an established drug in the long-term treatment of epilepsy. VPA has recently been shown in vivo to induce adult hippocampal neural progenitor cells to differentiate predominantly into neurons, mediated, at least in part, by the neurogenic transcription factor NeuroD (Hao, Creson et al. 2004; Hsieh, Nakashima et al. 2004).

Fluoxetine is an antidepressant whose mechanism of action has been shown to depend on hippocampal neurogenesis (Santarelli, Saxe et al. 2003).

VPA Treatment (test group 2): Adult Male Fisher 344 rats received two daily IP injections of 300 mg/kg VPA (experimental) or saline (control) for 28 days. VPA was also provided in the drinking water (12 g/liter) for the test group. Animals were imaged by MRI as described above.

Fluoxetine Treatment (test group 3): Adult Male Fisher 344 rats received daily oral gavage injections of 10 mg/kg Fluoxetine (experimental) or saline (control) for 28 days. Animals were imaged by MRI as described above.

Postmortem Analysis

To assess neurogenesis (neuronal proliferation, differentiation, and survival), the brains of animals from test and control groups were analyzed using quantitative analysis of fluorescent-labeled cells for specific markers (van Praag, Kempermann et al. 1999)

Following sacrifice, half of the brain were used to assess differentiation and cell survival by histology and immunohistochemistry using well established protocols. Data analysis was performed using stereology-based counting according to standard protocols with which BrainCells is familiar.

The remaining half of the brain was dissected further to isolate the hippocampus. The tissue was disrupted using a cell strainer and washed gently in cold 4% paraformaldehyde. Flow cytometry was then used to assess proliferation using Ki67 or Phospho H3 Ser10 as a marker.

By using half of the brain to assess differentiation and survival and the other half to investigate proliferation, it is possible to limit the number of animals required for the study which substantially reduced the costs (both animal costs and compound costs).

FACS Analysis Protocol

Hippocampal tissue was removed and placed on prewet cell strainer on a 50 ml falcon tube, and minced gently. Using a 3 cc syringe plunger, the cells were dispersed; the filter rinsed to get all cells. The cells were centrifuged and resuspended in 10 ml FACS buffer and counted and an aliquot removed (1-2×10⁷ cells) into a 5 ml FACS tube. The volume is brought to 5 ml with ice cold FACS buffer. Centrifuge, discard supernatant, resuspend in 5 ml ice cold FACS buffer, repeat centrifugation and finally, resuspend in a total volume of 1 ml of FACS buffer so that the cell concentration is 1-2×10⁶ per 100 μI. Add antibody or Propidium Iodide (PI) in a total volume of 30 μI to each reaction (usually 1 μg Ab/million cells). Include one tube with unlabeled cells and tubes with only one fluorophore used to set up FACS machine. Let sit on ice for 30 minutes. Add 2 ml ice cold FACS buffer. Centrifuge, discard supernatant carefully and then resuspend in 400 μl FACS buffer. Use immediately for analysis. Either an antibody to Phospho H3 Ser10 or Ki67 in the presence or absence of PI was used for the proliferation assays.

Histology Assay Protocol

Brains were postfixed overnight and then equilibrated in phosphate buffered 30% sucrose. Free floating 40 nm sections were collected on a freezing microtome and stored in cryoprotectant. Immunohistochemistry was performed as described in the subsequent section.

Immunohistochemistry Protocol

One half of the cryoprotected, frozen brain was coronally sectioned. Antibodies against BrdU and proteins of interest such as NeuN, neuronal and GFAP, astrocyte markers were also used for detection of cell differentiation. In brief, tissues were washed (0.01 M PBS), endogenous peroxidase blocked with 1% H₂O₂, and incubated in PBS (0.01 M, pH 7.4, 10% normal goat serum, 0.5% Triton X-100) for 2 hours at room temperature. Tissues were then incubated with primary antibody at 4° C. overnight. The tissues were then rinsed in PBS followed by incubation with biotinylated secondary antibody (1 hour, room temperature). Tissues were further washed with PBS and incubated in avidin-biotin complex kit solution at room temperature for 1 hour. Various fluorophores linked to streptavidin were used for visualization. Tissues were washed with PBS, briefly rinsed in dH₂O, serially dehydrated and coverslipped.

Cell Counting and Unbiased Stereology Protocol

This was limited to the hippocampal granule cell layer proper and a 50 μm border along the hilar margin that includes the neurogenic subgranule zone. The proportion of BrdU cells displaying a lineage-specific phenotype was determined by scoring the co-localization of cell phenotype markers with BrdU using confocal microscopy. Split panel and z-axis analysis were used for all counting. All counts were performed using multi-channel configuration with a 40× objective and electronic zoom of 2. When possible, 100 or more BrdU-positive cells were scored for each marker per animal. Each cell was manually examined in its full “z”-dimension and only those cells for which the nucleus was unambiguously associated with the lineage-specific marker were scored as positive. The total number of BrdU-labeled cells of each specific lineage (oligodendrocyte, astrocyte, neuron, other) per hippocampal granule cell layer and subgranule zone were determined using stained tissues. Overestimation was corrected using the Abercrombie method for nuclei with empirically determined average diameter of 13 μm within a 40 μm section.

Data Analysis

Once the data was acquired as described in the section entitled “CBV derivations with MRI” for CBV and in the section entitled “Postmortem analysis” for histology and FACS, the data was analyzed to determine if there was a statistically significant correlation between neurogenesis and CBV using within group analysis and between group analysis. Specifically, the cell counts generated using unbiased stereology and FACS were cross correlated with the signal change score obtained using MRI. Analyses included correlations between CBV changes (signal change score as a co-variant), proliferation, and lineage specific differentiation of BrdU labeled cells (e.g. total number of proliferating cells, total number of BrdU labeled cells, total number of dual labeled neuronal cells (neurogenesis), total number of dual labeled oligodendocytes, total number of dual labeled astrocytes) in a cross correlation between groups. Based on the preliminary results, an evaluation of other areas of the hippocampus was expanded to account for changes in CBV due to non-neurogenic causes versus neurogenic causes. Although results with exercise show that CA1 can be used to extract specific information about neurogenesis-induced changes in CBV, it is not certain that this region will be appropriate for drug-induced increases in CBV as a result of neurogenesis. Data analysis was performed in order to identify the optimal hippocampal region for analysis of non-neurogenesis as compared to neurogenesis induced changes in CBV.

### Vertebrate Animals

Description

Approximately 100 adult Male Fisher rats were used in these studies. Animals were subjected to different treatment protocols (control, exercise ad lib, treatment with valproic acid, or treatment with fluoxetane) as outlined in the Research Design and Methods section. At the onset and at the end of treatment, the animals were analyzed by MRI; upon completion of MRI analysis they were sacrificed. Neurogenesis in the animal brains was assessed by flow cytometry, histology and histochemical means.

Justification

Rats were used because this is the preferred species for screening CNS-acting drugs. Medline was searched to establish that there are no other mammalian species presently available for performing genetic and neuroscience behavioral-based evaluations as described in this proposal. In addition, the rat has been shown in multiple studies to be a good model for studying human disease, including human diseases with central nervous system abnormalities. The number of animals was chosen to generate enough variance to understand the series of complex relationships that connect CBV to neurogenesis.

Veterinary Care

All animal work took place at Columbia University, under the supervision of Dr. Dennis Kohn, D. V. M., Ph.D., who directs the animal care facility. Animals were watered, fed, and caged under NIH-approved guidelines, in a temperature and light-controlled environment with a 12/12-h light/dark cycle and provided food and water ad libitum. Animals were monitored daily by vivarium personnel for any signs or symptoms or discomfort. If animals began to show signs of weight loss or instability, they were examined by the Lab Animal Clinic veterinarian. Facilities were inspected regularly according to NIH guidelines.

Procedures

The rats received isoflurane to reduce movement and psychological anxiety while being imaged. Great care was taken to maintain the health and comfort of the rats while they were imaged. This fulfills both humanitarian as well as scientific goals. Many physiologic processes can influence MRI signal in the brain, particularly when measuring resting signal. For this reason a series of physiologic monitoring devices was purchased to allow for the tight monitoring of most physiologic measures while the mouse was being imaged. O₂ and CO₂ were continuously monitored with a micro-capnometer (Columbus Instruments); heart rate and pulse rate were continuously monitored using pulse oximetry (Model V33304, SergiVet). Temperature was continuously monitored with a thermistor (YSI Precision Thermometer 4000A). If needed, EKG and respiratory rate were recorded through devices built into the magnet.

Euthanasia

Rats were euthanized by an overdose of phenobarbital. This method is consistent with the recommendations of the Panel of Euthanasia of the American Veterinary Medical Association.

### REFERENCES FOR BACKGROUND OF THE INVENTION AND EXPERIMENTAL DETAILS I-III

Belliveau, J. W., B. R. Rosen, et-afrft99O). “Functional cerebral imaging by susceptibility-contrast NMR.” Magn Reson Med 14(3): 538-46.
Dunn, J. F., M. A. Roche, et al. (2004). “Monitoring angiogenesis in brain using steady-state quantification of DeltaR2 with MION infusion.” Magn Reson Med 51(1): 55-61.
Dunn, J. F., M. A. Roche, et al. (2003). “Steady-state MR imaging with MION for quantification of angiogenesis in normal brain and in brain tumors.” Adv Exp Med Biol 540: 221-6.
Eriksson, P. S., E. Perfilieva, et al. (1998). “Neurogenesis in the adult human hippocampus.” Nat Med 4(11): 1313-7.
Gold, S., B. Christian, et al. (1998). “Functional MRI statistical software packages: a comparative analysis.” Hum Brain Mapp 6(2): 73-84.
Gonzalez, R. G., A. J. Fischman, et al. (1995). “Functional MR in the evaluation of dementia: correlation of abnormal dynamic cerebral blood volume measurements with changes in cerebral metabolism on positron emission tomography with fludeoxyglucose F 18.” AJNR Am J Neuroradiol 16(9): 1763-70.
Hao, Y., T. Creson, et al. (2004). “Mood stabilizer valproate promotes ERK pathway-dependent cortical neuronal growth and neurogenesis.” J Neurosci 24(29): 6590-9.
Hsieh, J., J. B. Aimone, et al. (2004). “IGF-I instructs multipotent adult neural progenitor cells to become oligodendrocytes.” J Cell Biol 164(1): 111-22.
Hsieh, J., K. Nakashima, et al. (2004). “Histone deacetylase inhibition-mediated neuronal differentiation of multipotent adult neural progenitor cells.” Proc Natl Acad Sci USA 101(47): 16659-64.
Jiang, Q., Z. G. Zhang, et al. (2005). “Investigation of neural progenitor cell induced angiogenesis after embolic stroke in rat using MRI.” Neuroimage.
Kempermann, G. and F. H. Gage (2000). “Neurogenesis in the adult hippocampus.” Novartis Found Symp 231: 220-35; discussion 235-41, 302-6.
Kempermann, G., H. G. Kuhn, et al. (1997). “More hippocampal neurons in adult mice living in an enriched environment.” Nature 386(6624): 493-5.
Kempermann, G., H. G. Kuhn, et al. (1998). “Experience-induced neurogenesis in the senescent dentate gyrus.” J Neurosci 18(9): 3206-12.
Kola, I. and J. Landis (2004). “Can the pharmaceutical industry reduce attrition rates?” Nat Rev Drug Discov 3: 711.
Kuppusamy, K., W. Lin, et al. (1996). “In vivo regional cerebral blood volume: quantitative assessment with 3D T1-weighted pre- and postcontrast MR imaging.” Radiology 201(1): 106-12.
Lin, T. N., S. W. Sun, et al. (2002). “Dynamic changes in cerebral blood flow and angiogenesis after transient focal cerebral ischemia in rats. Evaluation with serial magnetic resonance imaging.” Stroke 33(12): 2985-91.
Lin, W., A. Celik, et al. (1999). “Regional cerebral blood volume: a comparison of the dynamic imaging and the steady state methods.” J Magn Reson Imaging 9(1): 44-52.
Lin, W., R. P. Paczynski, et al. (1997). “Quantitative measurements of regional cerebral blood volume using MRI in rats: effects of arterial carbon dioxide tension and mannitol.” Magn Reson Med 38(3): 420-8.
Louissaint, A., Jr., S. Rao, et al. (2002). “Coordinated interaction of neurogenesis and angiogenesis in the adult songbird brain.” Neuron 34(6): 945-60.
Mandeville, J. B., B. G. Jenkins, et al. (2001). “Regional sensitivity and coupling of BOLD and CBV changes during stimulation of rat brain.” Magn Reson Med 45(3): 443-7.
Morcuende, S., C. A. Gadd, et al. (2003). “Increased neurogenesis and brain-derived neurotrophic factor in neurokinin-1 receptor gene knockout mice.” Eur J Neurosci 18(7): 1828-36.
Moreno, H., F. Hua, et al. (2005). “Longitudinal mapping of mouse cerebral blood volume with MRI.” NMR in Biomedicine.
Newton, S. S. and R. S. Duman (2004). “Regulation of neurogenesis and angiogenesis in depression.” Curr Neurovasc Res 1(3): 261-7.
Palmer, T. D., J. Ray, et al. (1995). “FGF-2-responsive neuronal progenitors reside in proliferative and quiescent regions of the adult rodent brain.” Mol Cell Neurosci 6(5): 474-86.
Palmer, T. D., A. R. Willhoite, et al (2000). “Vascular niche for adult hippocampal neurogenesis.” J Comp Neurol 425(4): 479-94.
Ray, J., D. A. Peterson, et al. (1993). “Proliferation, differentiation, and long-term culture of primary hippocampal neurons.” Proc Natl Acad Sci USA 90(8): 3602-6.
Santarelli, L., M. Saxe, et al. (2003). “Requirement of hippocampal neurogenesis for the behavioral effects of antidepressants.” Science 301(5634): 805-9.
Small, S. A., M. K. Chawla, et al. (2004). “Imaging correlates of brain function in monkeys and rats isolates a hippocampal subregion differentially vulnerable to aging.” Proc Natl Acad Sci USA 101(18): 7181-6.
Small, S. A., W. Y. Tsai, et al. (2002). “Imaging hippocampal function across the human life span: is memory decline normal or not?” Ann Neurol 51(3): 290-5.
Small, S. A., E. X. Wu, et al. (2000). “Imaging physiologic dysfunction of individual hippocampal subregions in humans and genetically modified mice.” Neuron 28(3): 653-64.
Song, H. J., C. F. Stevens, et al. (2002). “Neural stem cells from adult hippocampus develop essential properties of functional CNS neurons.” Nat Neurosci 5(5): 438-45.
van Bruggen, N., E. Busch, et al. (1998). “High-resolution functional magnetic resonance imaging of the rat brain: mapping changes in cerebral blood volume using iron oxide contrast media.” J Cereb Blood Flow Metab 18(11): 1178-83.
van Praag, H., G. Kempermann, et al. (1999). “Running increases cell proliferation and neurogenesis in the adult mouse dentate gyrus.” Nat Neurosci 2(3): 266-70.
van Praag, H., A. F. Schinder, et al. (2002). “Functional neurogenesis in the adult hippocampus.” Nature 415(6875): 1030-4.
van Zijl, P. C, S. M. Eleff, et al. (1998). “Quantitative assessment of blood flow, blood volume and blood oxygenation effects in functional magnetic resonance imaging.” Nat Med 4(2): 159-67.
Wu, E. X., K. K. Wong, et al. (2003). “High-resolution in vivo CBV mapping with MRI in wild-type mice.” Magn Reson Med 49(4): 765-70.

### EXPERIMENTAL DETAILS IV

The hippocampal formation is a circuit made up of separate but interconnected hippocampal subregions (1). Among the multiple subregions that make up the hippocampal formation, the dentate gyrus (DG) is the only one that supports neurogenesis in the adult brain (2-5). A range of studies have established that physical exercise stimulates neurogenesis in the rodent hippocampus (6, 7) and enhances hippocampal-dependent cognition (8, 9). Furthermore, exercise has been shown to ameliorate age-related memory decline (7, 10-12), a process linked to dentate gyrus dysfunction (13, 14). Nevertheless, whether exercise stimulates neurogenesis in humans remains unknown.

With this question in mind, different imaging approaches that might provide an in vivo correlate of neurogenesis have been explored. Although imaging radioligands designed to bind newly dividing cells is an attractive approach, PET (positron emission tomography) imaging suffers inherently poor resolution and cannot visualize the dentate gyrus. Additionally, radiolabelling newborn cells introduces potential safety concerns. For these reasons, the use of MRI (magnetic resonance imaging) technologies is preferred. In this regard, the tight coupling between neurogenesis and angiogenesis (15, 16), and the fact that angiogenesis gradually gives rise to new blood vessels (17, 18), ultimately increasing regional cerebral blood volume (CBV) (19-21), is striking. Because CBV can be measured with MRI, it was hypothesized that a regionally selective increase in hippocampal CBV might provide an imaging correlate of neurogenesis.

This hypothesis was first tested in exercising mice, in whom parallel in vivo and in vitro studies can be performed. Because of the importance of tracking longitudinal changes in CBV, newly developed MRI approach (22) was optimized so that hippocampal CBV maps could be generated repeatedly and safely over time. Once confirmed in mice, tests were performed to determine whether the in vivo correlate of neurogenesis can be observed in exercising humans, optimizing an MRI approach (23, 24) previously shown capable of generating hippocampal CBV maps in non-human primates (13).

### Methods

Exercise

Mice: 46 C57BL/6 mice, 7 weeks old, were used: 23 exercising and 23 non-exercising animals. The experimental mice were placed in cages with running wheels (Lafayette Instrument Company). The animals ran voluntarily for 2 weeks. MRI images were acquired at the following time points: week 0 (baseline), week 2 (when exercise was stopped), week 4 and week 6. The thymidine analog bromodeoxyuridine (BrdU) marker was injected intraperitoneally for 7 consecutive days (60 mg/kg/day) during the second week of the experiment. At week 6 the animals were anesthetized and sacrificed in accordance with institutional guidelines.

Human: Subjects were recruited who fulfilled AHA (American Heart Association) criteria for below average aerobic fitness (VO2max<43 for men, <37 for women) (39). The 11 enrolled subjects engaged in an exercise training protocol for 12 weeks at Columbia University Fitness Center, at a frequency of four times a week. Each exercise session lasted about one hour: 5 min low intensity warm-up on a treadmill or stationary bicycle; 5 min stretching; 40 min aerobic training; 10 min cool down and stretching. During the 40 min of aerobic activity, subjects were permitted to select from cycling on a stationary ergometer, running on a treadmill, climbing on a stairmaster or using an elliptical trainer.

VO₂max (maximum volume of oxygen consumption) was measured by a graded exercise test on an Ergoline 800S electronic-braked cycle ergometer (SensorMedics Corp., Anaheim, Calif.). Each subject began exercising at 30 watts (W) for 2 min, and the work rate was continually increased by 30 W each 2 min until VO2max criteria (RQ of 1.1 or >, increases in ventilation without concomitant increases in VO₂, maximum age-predicted heart rate is reached and or volitional fatigue) was reached. Minute ventilation was measured by a pneumotachometer connected to a FLO-1 volume transducer module (PHYSIO-DYNE Instrument Corp., Quogue, N.Y.). Percentages of expired oxygen (O₂) and carbon dioxide (CO₂) were measured using a paramagnetic O₂ and infrared CO₂ analyzers connected to a computerized system (MAX-1, PHYSIO-DYNE Instrument Corp., Quogue, N.Y.). These analyzers were calibrated against known medical grade gases. The highest VO₂ value attained during the graded exercise test is considered VO₂max.

In Vivo Imaging

Mice: Mice were imaged with a 9.4 tesla Bruker scanner (AVANCBV 400WB spectrometer, Bruker NMR, Inc., Billerica, Mass.), following the protocol as previously described (22). Briefly, axial T2-weighted images were optimally acquired with a fast sequence (TR/TEeff=2000 ms/70 ms; 30 mm-i.d. birdcage RF probe; shielded gradient system=100 G/cm; rapid acquisition with relaxation enhancement (RARE) factor=16; FOV=19.6 mm; acquisition matrix=256×256; 8 slices; slice thickness=0.6 mm, slice gap=0.1 mm; NEX=28). Five sets of images were acquired sequentially, each requiring 16 min. The first two sets were pre-contrast. Gadodiamide was then injected I.P. (13 mmol/kg) through a catheter placed intraperitoneally before imaging. The last three sets corresponded to the post-contrast images. To prevent head motion and reduce anxiety, the animals were anesthetized with isofluorane gas (1.5 vol % for maintenance at 1 L/min air flow) via a nose cone. Isofluorane was chosen because it induces minimal cerebral hemodynamic change (40). Monitoring of the heart rate, respiratory rate and SaO₂ was performed during the whole procedure. Relative CBV was mapped as changes of the transverse relaxation rate (ΔR2) induced by the contrast agent. When the contrast agent reaches uniform distribution, CBV maps can be measured from steady-state T2-weighted images as: CBV∞ΔR2=ln(Spre/Spost)/TE; where TE=effective echo time; Spre=signal before the contrast administration; Spost=signal after the contrast agent reaches steady-state. To control for differences in levels of contrast administration, cardiac output, and global blood flow, the derived maps were normalized to the maximum 4 pixels signal value of the posterior cerebral vein. Visualized anatomical landmarks were used together with standard atlases to identify the localization of four hippocampal subregions: the dentate gyrus, the CA3 subfield, the CA1 subfield and the entorhinal cortex (41). The normalized CBV measurements from each subregion were used for group data analysis.

Human: Subjects were imaged with a 1.5 tesla scanner Philips Intera scanner. As previously described (13), coronal T1-weighted images (repetition time, 20 ms; echo time, 6 ms; flip angle, 25 degrees; in plane resolution, 0.86 mm×86 mm; slice thickness, 4 mm) were acquired oriented perpendicular to the hippocampal long-axis before and 4 min. after i.v. administration of the contrast gadolinium (0.1 mmol/kg). The difference between pre-contrast and post-contrast images was used to access the regional CBV map. To control for differences in levels of contrast administration, cardiac output, and global blood flow, the derived differences in signal intensity were normalized to the maximum 4 pixels signal value of the sagittal sinus (24). For each subject, the precontrast scan was used to identify the slice with the best visualization of the external morphology and internal architecture of the hippocampal formation. Visualized anatomical landmarks were used together with standard atlases to identify the general locale of four subregions: the dentate gyrus, the CA1 subfield, the subiculum and the entorhinal cortex (13). The normalized CBV measurements from each subregion were used for group data analysis.

Microscopy

Immunohistochemistry: Free-floating 40-μm coronal sections were used in the determination of BrdU labeling. DNA denaturation was conducted by incubation for 1 hr at 2N HCl at 37° C., followed by washing in boric buffer (pH 8.5). After washing, sections were incubated for 30 min in 10% H₂O₂ to eliminate endogenous peroxidases. After blocking with 3% normal donkey serum in 0.2% Triton X-100, sections were incubated with monoclonal anti-BrdU (1:600; Roche) overnight at 4° C. Sections were then incubated for 1 hr at room temperature (RT) with the secondary antibody (biotinylated donkey anti-mouse; Jackson Immuno Research Lab) followed by amplification with an avidin-biotin complex (Vector Laboratories), and visualized with DAB (Sigma). For double-immunolabelling, free-floating sections were incubated in a mixture of primary antibodies, anti-BrdU (1:600; Roche) and anti-NeuN (1:500; Chemicon), raised in different species for overnight. For visualization, Alexa Fluor-conjugated appropriate secondary antibodies (1:300; Molecular Probes) raised in goat were used for 1 hour at room temperature. Blocking serum and primary and secondary antibodies were applied in 0.2% Triton X-100 in PBS. Sections for fluorescent microscopy were mounted on slides in Vectashield (Vector Lab). For control of the specificity of immunolabelling, primary antibodies were omitted and substituted with appropriate normal serum. Slides were viewed using confocal microscope (Nikon E800, BioRad 2000). The images presented are stacks of 6-16 optical sections (step 1 mm) that were collected individually (in the green and red channels) or simultaneously with precaution against cross-talk between channels. They were processed with Adobe Photoshop 7.0 without contrast and brightness changes in split images.

Quantitation of BrdU labeling: Every sixth section throughout the hippocampus was processed for BrdU immunohistochemistry. Ten sections were used for each animal. All BrdU-labeled cells in the dentate gyrus (granule cell layer and at a distance less than 60 μm from it) were counted under a light microscope by an experimenter blinded to the study code. The total number of BrdU-labeled cells per section was determined and multiplied by the number of sections obtained from each animal to achieve the total number of cells per dentate gyrus.

Cognitive Testing

Declarative memory was measured with a version of the Rey Auditory Verbal Learning Test (29) modified to increase variability in memory performance among healthy young adults. Twenty non-semantically or phonemically related words were presented over three learning trials, in which the test administrator read the word list and the subject free recalled as many words as possible. Administration of the three learning trials was immediately followed by one learning trial of a distracter list and then a short delayed free recall of the initial list. After a 90-min delay period, subjects were asked to freely recall words from the initial list and then to freely recall items from the distracter list. After a 24-hour delay period, subjects were contacted by telephone and asked to freely recall items from the initial list and then from the distracter list. They were then administered a forced-choice recognition trial in which they were required to identify the 20 words from the initial learning trial among semantically and phonemically related words as well as words from the distracter trial. Finally, a source memory trial was administered in which subjects were read a list containing only words from the initial learning list and from the distracter list and were asked to identify from which list each word came. Two forms of the verbal learning test were created and the administration order was counterbalanced. As in previous studies (42), words correctly recalled on the first trial of the initial learning trials, the average number of words recalled across the three learning trials, the number of words from the initial learning trial that were correctly recalled after a short delay (<5 min), the number of words from the initial learning trial that were correctly recalled after a 90-min delay, the number of items correctly identified on the recognition trial, and the correct number of items identified on the source memory trial were measured.

### Results

Selective Increases in Dentate Gyrus CBV Provide an In Vivo Correlate of Exercise-Induced Neurogenesis

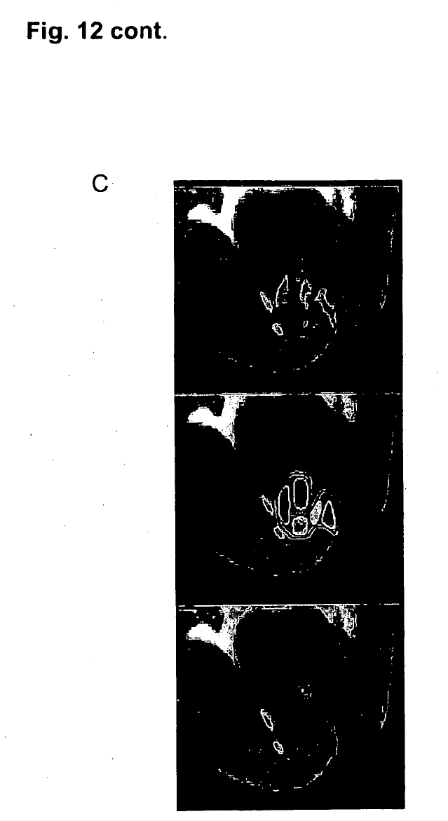

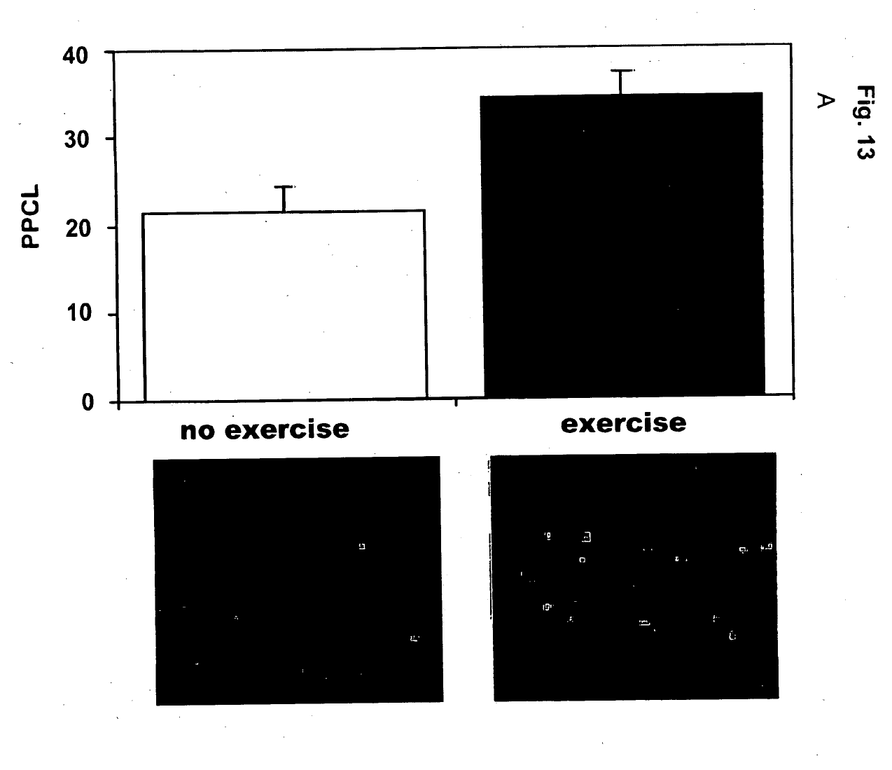

The design of the experimental protocol (FIG. 12a) was guided by the observation that angiogenesis-induced sprouting of new blood vessels progresses through different stages, forming gradually over time (18). Accordingly, mice were allowed to exercise for 2 weeks, the period during which neurogenesis reaches its maximum increase, and BrdU (bromo-deoxyuridine), a marker of newly born cells, was injected daily during the second week. To capture the predicted delayed effect in CBV, mice were kept alive for 4 more weeks, then sacrificed and processed for BrdU labeling. Hippocampal CBV maps were generated four times over the 6-week experiment: at pre-exercise baseline and at week 2, week 4, and week 6. A control group was imaged in parallel, following the identical protocol but without exercise. The hippocampal formation is made up of multiple interconnected subregions, including the entorhinal cortex, the dentate gyrus, the CA1 and CA3 subfields, and the subiculum. CBV measurements were reliably extracted from all hippocampal subregions except the subiculum (FIG. 12c).

A repeated-measures ANOVA was used to analyze the imaging dataset. A group X time interaction was found only for the dentate gyrus, showing that exercise was associated with a selective increase in dentate gyrus CBV (F=5.0, p=0.034). As shown by simple contrasts, the effect was driven by a maximum increase that emerged 2 weeks after the cessation of exercise, from week 2 to week 4 (F=5.9, p=0.021) (FIG. 12b). The entorhinal cortex was the only other hippocampal subregion whose CBV increased appreciably over time, although not achieving statistical significance (FIG. 12b). Although exercise might potentially affect CBV by increasing metabolism and cerebral blood flow, previous studies (25, 26) have shown that exercise-induced changes in metabolism should manifest during, not after, the exercise regimen. Thus, the observed spatiotemporal profile with which CBV emerged fits better with a model of exercise-induced angiogenesis (18) in the dentate gyrus (FIG. 12a).

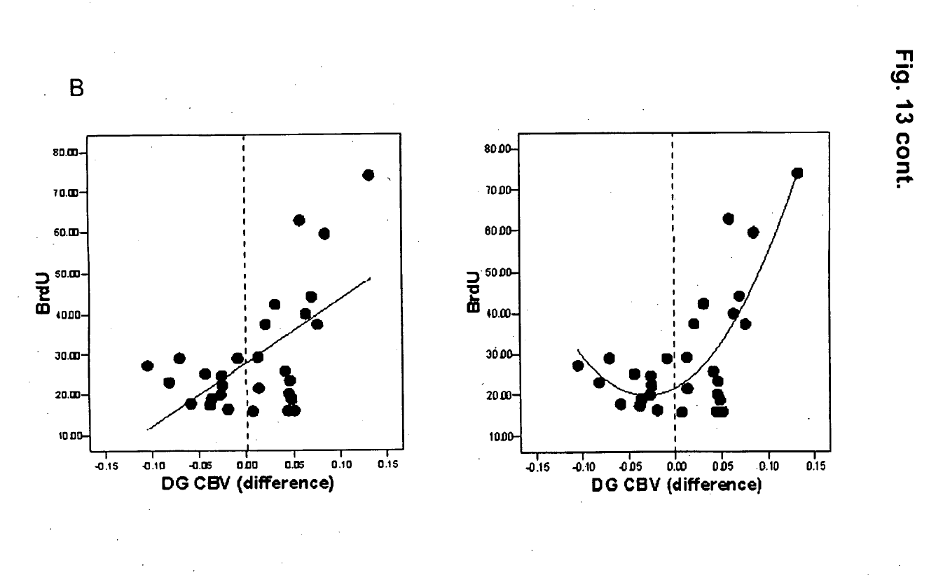

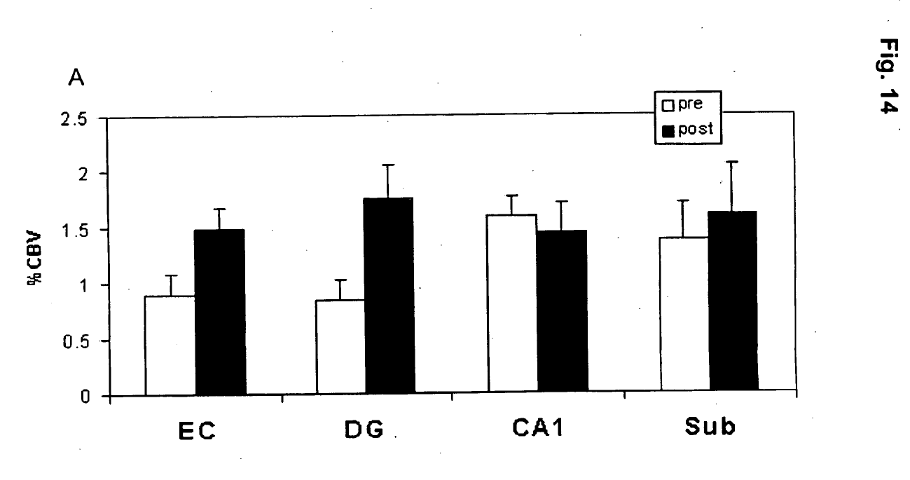

In agreement with previous studies (6), the exercise group was found to have greater BrdU labeling compared to the non-exercise group (F=9.8; p=0.004) (FIG. 13a). Over 90% of BrdU-positive cells co-labelled for NeuN, a neuron-specific marker (FIG. 13a). To examine the relationship between neurogenesis and CBV, the repeated-measures model was again used including BrdU as a covariate. A significant time X BrdU interaction was observed only for dentate gyrus CBV (F=3.3, p=0.039), driven primarily by changes from week 2 to week 4 (F=8.8, p=0.006). As shown by a direct analysis, this effect reflected a positive correlation between BrdU and changes in CBV from week 2 to week 4 (beta=0.58, p=0.001) (FIG. 13b). Of note, when BrdU was included as a covariate in the ANOVA, the group X time effect observed in the dentate gyrus was no longer significant, confirming that neurogenesis accounted for the exercise effect on CBV. Visual inspection of the relationship between changes in dentate gyrus CBV and BrdU (FIG. 13b) suggested that a quadratic vs. a linear model better characterized the relationship, which was confirmed by curve estimation analysis (linear model, R-squared=0.34, p=0.001; quadratic model, R-square=0.59, p<0.0001). Thus, the association between changes in dentate gyrus CBV and BrdU exists primarily when CBV increases with exercise (FIG. 13b).

Selective Increases in Dentate Gyrus CBV Observed in Exercising Humans

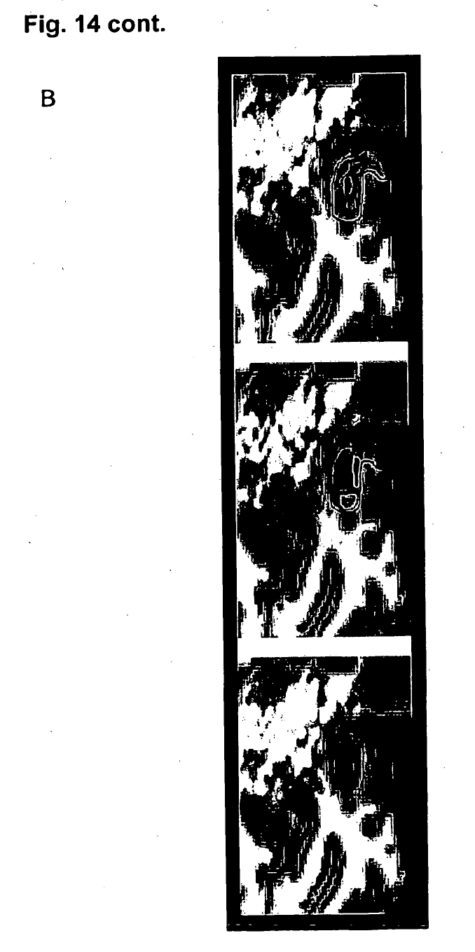

Once it was established that dentate gyrus CBV provides a correlate of exercise-induced neurogenesis, there was interest in testing whether this effect is observed in exercising humans. CBV maps of the human hippocampal formation were generated using the previously reported MRI approach, specifically tailored for imaging the primate hippocampal formation (13). Eleven subjects (mean age=33) participated in the study, completing a 3-month aerobic exercise regimen in which hippocampal CBV maps were generated before and after exercise. CBV values were reliably measured for all hippocampal subregions, except the CA3 subregion (FIG. 14b). Compared to experimental animals, in humans it is impossible to control the inter-individual differences in physical activity performed during daily life. Therefore, before and after exercise we measured VO₂max (maximum volume of oxygen consumption), the gold standard measure of exercise-associated aerobic fitness (27, 28) to quantify individual differences in degree of exercise. Cognitive performance was assessed using a modified Rey Auditory Verbal Learning Test (RAVLT) (29), whose design allows cognition to be tested across different learning trials and during delayed recall, recognition, and source memory. Ten subjects were cognitively assessed after exercise, 8 of which were assessed at pre-exercise baseline.

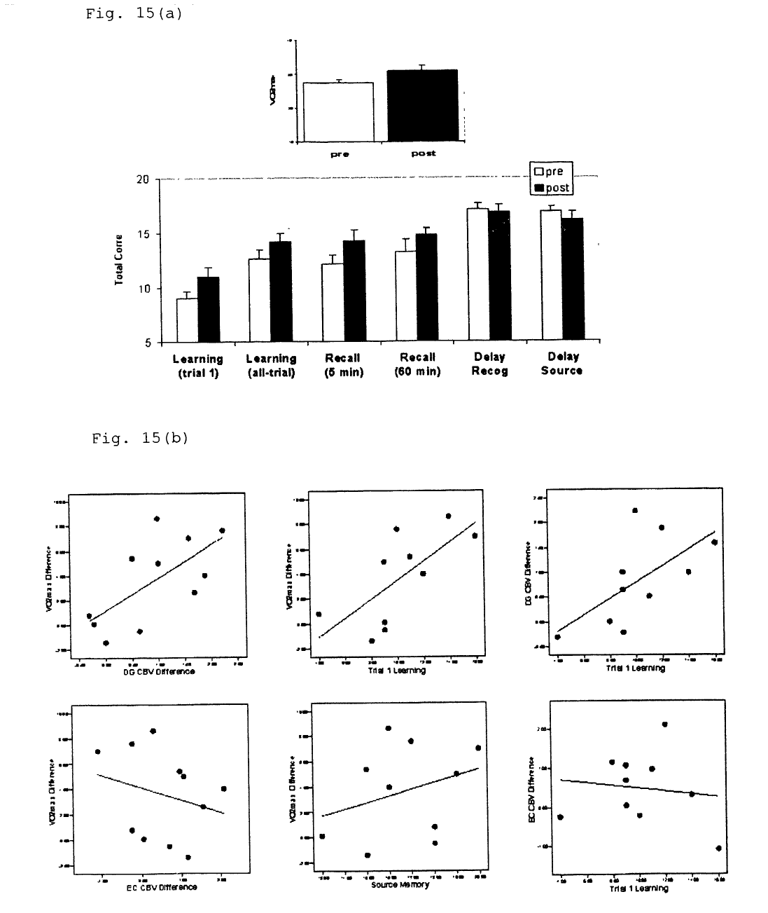

A repeated-measures ANOVA used to analyze the imaging data showed that the dentate gyrus was the only hippocampal subregion whose CBV significantly increased over time (F=12, p=0.006) (FIG. 14a). As in mice, the entorhinal cortex was the only other hippocampal subregion whose CBV increased appreciably over time, although not achieving statistical significance, (F=4.3, p=0.064) (FIG. 14a). As a group, VO₂max values significantly increased over time (F=11.6; p=0.007) (FIG. 15a) and to confirm that the imaged changes were directly related to exercise and not simply caused by a test-retest effect, it was found that individual differences in dentate gyrus CBV were correlated to individual changes in VO₂max (beta=0.662, p=0.027) (FIG. 15b). Importantly, a correlation between CBV and VO₂max was not observed for any other hippocampal subregion, including the entorhinal cortex (FIG. 15b) confirming that exercise has a selective effect on dentate gyrus CBV.

Cognitively, individuals performed significantly better on trial 1 learning (F=7.0, p=0.027) post-exercise, with a trend toward improvement on all-trial learning (F=5.0, p=0.053) and delayed recall (F=5.0, p=0.057). There was no effect on delayed recognition (F=0.19, p=0.67) or source memory (F=0.15, p=0.25) (FIG. 15a). To test that cognitive improvement was related to exercise per se, it was found that individual changes in trial 1 learning were correlated with individual changes in VO₂max (beta=0.660, p=0.037). However, because only 8 of the 10 subjects completed pre-exercise cognitive testing, the analysis was repeated using post-exercise cognitive performance scores. Again, it was found that changes in VO₂max correlated exclusively with post-exercise trial 1 learning (beta=0.70, p=0.026) (FIG. 15b). Additional analyses showed that the correlation between changes in VO₂max and cognition was selective to trial 1 learning (FIG. 15b), thereby confirming that, despite apparent increases in other cognitive tasks, this particular ability was selectively influenced by exercise.

Finally, the relationship between cognition and CBV was examined. Among all hippocampal subregions, the correlation between improvements in trial 1 performance and increases in dentate gyrus CBV was the only one that trended toward significance (beta=0.62, p=0.052). Because of the missing pre-exercise data, all the analyses comparing changes in CBV with post-exercise cognition were repeated, finding an exclusive correlation between post-exercise trial 1 learning and dentate gyrus CBV (beta=0.63, p=0.026) (FIG. 15b).

### Discussion

The results of these studies show that dentate gyrus CBV is an imaging correlate of exercise-induced neurogenesis and that this correlate is expressed in exercising humans. As with any imaging biomarker, testing it against an in vitro measure of neurogenesis is not currently possible in humans. Nevertheless, the remarkably similar effect exercise had on hippocampal CBV in both humans and mice suggest similar underlying mechanisms. Moreover, rodent studies have shown that individual differences in degree of exercise correlate with levels of neurogenesis (30), results that parallel human findings in whom individual differences in degree of exercise correlated with levels of CBV. Taken together, the findings provide support for the hypothesis that, as in mice, exercise stimulates neurogenesis in humans.

Exercise has been shown to have a pleiotropic effect on the brain (31, 32), ameliorating age-related cognitive decline (7, 10-12) and improving depression (33, 34). Studies in humans (14, 35), non-human primates (13, 36), and rodents (13) have suggested that the dentate gyrus is a hippocampal subregion particularly vulnerable to the aging process, and dentate gyrus dysfunction has been linked to cognitive aging (13, 14). By finding that humans express an exercise-induced correlate of neurogenesis and by optimizing the tools that established the cross-species biomarker, future studies can now gain deeper insight into the functional significance of neurogenesis in both the normal and aging brain. Furthermore, the imaging tools presented here are uniquely suited to investigate potential pharmacological modulators of neurogenesis, testing their role in treating depression (37) or in reversing the cognitive decline that occurs in all of us as we age (7, 38).

### REFERENCES FOR EXPERIMENTAL DETAILS IV

1. Amaral, D. G. & Witter, M. P. (1989) Neuroscience 31, 571-91.
2. Altman, J. & Das, G. D. (1965) J Comp Neurol 124, 319-35.
3. Kaplan, M. S. & Hinds, J. W. (1977) Science 197, 1092-4.
4. Kempermann, G., Kuhn, H. G. & Gage, F. H. (1997) Nature 386, 493-5.
5. Eriksson, P. S., Perfilieva, E., Bjork-Eriksson, T., Alborn, A. M., Nordborg, C., Peterson, D. A. & Gage, F. H. (1998) Nat Med 4, 1313-7.
6. van Praag, H., Christie, B. R., Sejnowski, T. J. & Gage, F. H. (1999) Proc Natl Acad Sci USA 96, 13427-31.
7. van Praag, H., Shubert, T., Zhao, C. & Gage, F. H. (2005) J Neurosci 25, 8680-5.
8. Snyder, J. S., Hong, N. S., McDonald, R. J. & Wojtowicz, J. M. (2005) Neuroscience 130, 843-52.
9. Shors, T. J., Miesegaes, G., Beylin, A., Zhao, M., Rydel, T. & Gould, E. (2001) Nature 410, 372-6.
10. Kramer, A. F., Hahn, S., Cohen, N. J., Banich, M. T., McAuley, E., Harrison, C. R., Chason, J., Vakil, E., Bardell, L., Boileau, R. A. & Colcombe, A. (1999) Nature 400, 418-9.
11. Chan, A. S., Ho, Y. C., Cheung, M. C., Albert, M. S., Chiu, H. F. & Lam, L. C. (2005) J Am Geriatr Soc 53, 1754-60.
12. Weuve, J., Kang, J. H., Manson, J. E., Breteler, M. M., Ware, J. H. & Grodstein, F. (2004) Jama 292, 1454-61.
13. Small, S. A., Chawla, M. K., Buonocore, M., Rapp, P. R. & Barnes, C. A. (2004) Proc Natl Acad Sci USA 101, 7181-6.
14. Small, S. A., Tsai, W. Y., DeLaPaz, R., Mayeux, R. & Stern, Y. (2002) Ann Neurol 51, 290-5.
15. Palmer, T. D., Willhoite, A. R. & Gage, F. H. (2000) J Comp Neurol 425, 479-94.
16. Louissaint, A., Jr., Rao, S., Leventhal, C. & Goldman, S. A. (2002) Neuron 34, 945-60.
17. Carmeliet, P. (2000) Nat Med 6, 389-95.
18. Carmeliet, P. (2003) Nat Med 9, 653-60.
19. Dunn, J. F., Roche, M. A., Springett, R., Abajian, M., Merlis, J., Daghlian, C. P., Lu, S. Y. & Makki, M. (2004) Magn Reson Med 51, 55-61.
20. Pathak, A. P., Schmainda, K. M., Ward, B. D., Linderman, J. R., Rebro, K. J. & Greene, A. S. (2001) Magn Reson Med 46, 735-47.
21. Jiang, Q., Zhang, Z. G., Ding, G. L., Zhang, L., Ewing, J. R., Wang, L., Zhang, R., Li, L., Lu, M., Meng, H., Arbab, A. S., Hu, J., Li, Q. J., Pourabdollah Nejad, D. S., Athiraman, H. & Chopp, M. (2005) Neuroimage 28, 698-707.
22. Moreno, H., Hua, F., Brown, T. & Small, S. (2006) NMR Biomed.
23. Lin, W., Paczynski, R. P., Kuppusamy, K., Hsu, C. Y. & Haacke, E. M. (1997) Magn Reson Med 38, 420-8.
24. Lin, W., Celik, A. & Paczynski, R. P. (1999) J Magn Reson Imaging 9, 44-52.
25. Vissing, J., Andersen, M. & Diemer, N. H. (1996) J Cereb Blood Flow Metab 16, 729-36.
26. Ide, K. & Secher, N. H. (2000) Prog Neurobiol 61, 397-414.
27. Mitchell, J. H., Sproule, B. J. & Chapman, C. B. (1958) J Clin Invest 37, 538-47.
28. Wagner, P. D. (1996) Annu Rev Physiol 58, 21-50.
29. Rey, A. (1964) L'examen clinique en psychologie (Presses Universitaires de France, Paris).
30. van Praag, H., Kempermann, G. & Gage, F. H. (1999) Nat Neurosci 2, 266-70.
31. Cotman, C. W. & Berchtold, N. C. (2002) Trends Neurosci 25, 295-301.
32. Dishman, R. K., Berthoud, H. R., Booth, F. W., Cotman, C. W., Edgerton, V. R., Fleshner, M. R., Gandevia, S. C., Gomez-Pinilla, F., Greenwood, B. N., Hillman, C. H., Kramer, A. F., Levin, B. E., Moran, T. H., Russo-Neustadt, A. A., Salamone, J. D., Van Hoomissen, J. D., Wade, C. E., York, D. A. & Zigmond, M. J. (2006) Obesity (Silver Spring) 14, 345-56.
33. Dunn, A. L., Trivedi, M. H., Kampert, J. B., Clark, C. G. & Chambliss, H. O. (2005) Am J Prev Med 28, 1-8.
34. Blumenthal, J. A., Babyak, M. A., Moore, K. A., Craighead, W. E., Herman, S., Khatri, P., Waugh, R., Napolitano, M. A., Forman, L. M., Appelbaum, M., Doraiswamy, P. M. & Krishnan, K. R. (1999) Arch Intern Med 159, 2349-56.
35. West, M. J., Coleman, P. D., Flood, D. G. & Troncoso, J. C. (1994) Lancet 344, 769-72.
36. Gazzaley, A. H., Siegel, S. J., Kordower, J. H., Mufson, E. J. & Morrison, J. H. (1996) Proc Natl Acad Sci USA 93, 3121-5.
37. Santarelli, L., Saxe, M., Gross, C., Surget, A., Battaglia, F., Dulawa, S., Weisstaub, N., Lee, J., Duman, R., Arancio, O., Belzung, C. & Hen, R. (2003) Science 301, 805-9.
38. Small, S. A., Stern, Y., Tang, M. & Mayeux, R. (1999) Neurology 52, 1392-6.
39. Fletcher, G. F., Balady, G., Froelicher, V. F., Hartley, L. H., Haskell, W. L. & Pollock, M. L. (1995) Circulation 91, 580-615.
40. Lei, H., Grinberg, O., Nwaigwe, C. I., Hou, H. G., Williams, H., Swartz, H. M. & Dunn, J. F. (2001) Brain Res 913, 174-9.
41. Paxinos, G. & Franklin, K. (2001) The mouse brain in stereotaxic coordinates (Academic Press.
42. Van der Elst, W., van Boxtel, M. P., van Breukelen, G. J. & Jolles, J. (2005) J Int Neuropsychol Soc 11, 290-302.

1. A method for treating a mammalian subject afflicted with a disorder associated with reduced neurogenesis in the subject's hippocampal dentate gyrus which comprises administering to the subject a therapeutically effective amount of a compound which increases cerebral blood volume in the subject's hippocampal dentate gyrus by a percentage greater than that by which it increases the cerebral blood volume in the subject's hippocampal CA1 region, thereby treating the subject.

2. The method of claim 1, wherein the subject is a human.

3. The method of claim 2, wherein the disorder is selected from the group consisting of Alzheimer's disease, post-traumatic stress syndrome, age-related memory loss and depression.

4. The method of claim 3, wherein the disorder is Alzheimer's disease.

5. The method of claim 3, wherein the disorder is post-traumatic stress syndrome.

6. The method of claim 3, wherein the disorder is age-related memory loss, and the subject is older than 65-years old.

7. The method of claim 3, wherein the disorder is depression.

8. The method of claim 1, wherein the compound is a serotonin-selective uptake inhibitor.

9. A method for inhibiting the onset in a mammalian subject of a disorder associated with reduced neurogenesis in the subject's hippocampal dentate gyrus which comprises administering to the subject a prophylactically effective amount of a compound which increases cerebral blood volume in the subject's hippocampal dentate gyrus by a percentage greater than that by which it increases the cerebral blood volume in the subject's hippocampal CA1 region, thereby inhibiting the onset of the disorder.

10. The method of claim 9, wherein the subject is a human.

11. The method of claim 10, wherein the disorder is selected from the group consisting of Alzheimer's disease, post-traumatic stress syndrome, age-related memory loss and depression.

12. The method of claim 11, wherein the disorder is Alzheimer's disease.

13. The method of claim 11, wherein the disorder is post-traumatic stress syndrome.

14. The method of claim 11, wherein the disorder is age-related memory loss and the subject is older than 65-years old.

15. The method of claim 11, wherein the disorder is depression.

16. The method of claim 9, wherein the compound is a serotonin-selective uptake inhibitor.

17. A method for treating a mammalian subject afflicted with a disorder associated with increased neurogenesis in the subject's hippocampal dentate gyrus which comprises administering to the subject a therapeutically effective amount of a compound which decreases cerebral blood volume in the subject's hippocampal dentate gyrus by a percentage greater than that by which it decreases the cerebral blood volume in the subject's hippocampal CA1 region, thereby treating the subject.

18. The method of claim 17, wherein the subject is human.

19. A method of claim 18, wherein the disorder is epilepsy.

20. A method for inhibiting the onset in a mammalian subject of a disorder associated with increased neurogenesis in the subject's hippocampal dentate gyrus which comprises administering to the subject a prophylactically effective amount of a compound which decreases cerebral blood volume in the subject's hippocampal dentate gyrus by a percentage greater than that by which it decreases the cerebral blood volume in the subject's hippocampal CA1 region, thereby inhibiting the onset of the disorder.

21-22. (canceled)

23. A method for determining whether an agent increases neurogenesis in a mammalian subject's hippocampal dentate gyrus which comprises:
(a) determining the cerebral blood volume of a volume of tissue in the subject's hippocampal dentate gyrus and of a volume of tissue in the subject's hippocampal CA1 region;
(b) administering the agent to the subject in a manner permitting it to enter the subject's hippocampal dentate gyrus and hippocampal CA1 regions;
(c) after a period of time sufficient to permit a detectable increase in neurogenesis in the subject's hippocampal dentate gyrus by an agent known to cause such an increase, determining the cerebral blood volume of the volume of tissue in the subject's hippocampal dentate gyrus and the volume of tissue in the subject's hippocampal CA1 region; and
(d) comparing the cerebral blood volumes determined in steps (a) and (c) to determine whether a neurogenesis-specific increase in cerebral blood volume has occurred in the subject's hippocampal dentate gyrus, such increase indicating that the agent increases neurogenesis in the subject's hippocampal dentate gyrus.

24-30. (canceled)

31. A method for determining whether an agent decreases neurogenesis in a mammalian subject's hippocampal dentate gyrus which comprises:
(a) determining the cerebral blood volume of a volume of tissue in the subject's hippocampal dentate gyrus and a volume of tissue in the subject's hippocampal CA1 region;
(b) administering the agent to the subject in a manner permitting it to enter the subject's hippocampal dentate gyrus and hippocampal CA1 regions;
(c) after a period of time sufficient to permit a detectable decrease in neurogenesis in the subject's hippocampal dentate gyrus by an agent known to cause such a decrease, determining the cerebral blood volume of the volume of tissue in the subject's hippocampal dentate gyrus and the volume of tissue in the subject's hippocampal CA1 region; and
(d) comparing the cerebral blood volumes determined in steps (a) and (c) to determine whether a neurogenesis-specific decrease in cerebral blood volume has occurred in the subject's hippocampal dentate gyrus, such decrease indicating that the agent decreases neurogenesis in the subject's hippocampal dentate gyrus.

32-37. (canceled)
